(19) United States
(12) Patent Application Publication
(10) Pub. No.: US 2002/0032176 A1
(43) Pub. Date: Mar. 14, 2002

(54) CARCINOGENESIS INHIBITORS

(76) Inventors: TAKASHI MAOKA, SHIGA (JP); KOOICHI MOCHIDA, OSAKA (JP); MUTSUO KOZUKA, KYOTO (JP); HARUKUNI TOKUDA, KYOTO (JP); YOSHIHIRO ITO, KYOTO (JP); YOKO OKUDA, HYOGO (JP)

Correspondence Address: WOOD PHILLIPS VAN SANTEN, CLARK & MORTIMER, 500 WEST MADISON STREET, SUTIE 3800, CHICAGO, IL, 60661

(21) Appl. No.: 09/331,747

(22) PCT Filed: Dec. 24, 1997
(86) PCT No.: PCT/JP97/04765

(30) Foreign Application Priority Data
Dec. 25, 1996 (JP) 8-357313
Jan. 21, 1997 (JP) 9-23344
Sep. 25, 1997 (JP) 9-279885
Sep. 25, 1997 (JP) 9-279886
Nov. 12, 1997 (JP) 9-329594

(51) Int. Cl. A61K 31/665; A01N 57/00

(52) U.S. Cl. 514/100

(57) ABSTRACT

Carcinogenesis inhibitors containing as the active ingredient carotenoids extracted from the pure-line species of paradicsom paprika (species classified as Capsicum annuum L. var. grossum), etc., such as capsanthin, its fatty acid esters, capsorubin diesters, capsanthin 3,6-epoxide, capsorubin and cucurbitaxanthine A-3′ ester. These carcinogenesis inhibitors and paradicsom paprika extracts originate in natural substances and, therefore, make it possible to provide excellent Epstein-Barr virus genome inactivating agents. Thus, they are expected as being useful in preventing carcinogenesis and, based on their effects, applicable in various fields including drugs, cosmetics and health foods.

### FIELD OF THE INVENTION

This invention relates to carcinogenesis inhibitors. More specifically, this invention relates to carcinogenesis inhibitors comprising carcinogenesis inhibitive plant extracts and carotenoids obtained from those plant extracts as active ingredients.

### DESCRIPTION OF THE PRIOR ART

Cancer is the leading cause of death of the Japanese, and prevention of cancer is of critical importance for the national health problems. In general, cancer is considered to develop proceeding two different stages, initiation and promotion. That is, DNA of genes of normal cells is irreversibly damaged by initiators such as ultraviolet rays, radioactive rays or mutagens (initiation), and latent cancer cells generated as a result of the initiation are repeatedly irritated by chemical substances (promotion) to transform malignantly. Accordingly, cancer is preventable by interrupting either initiation or promotion.

However, it is impossible to remove initiators completely due to the existence of ultraviolet rays, radiation and other multagens in our environment. Simultaneously, most adults are already considered to hold initiated cells so that interrupting initiation is not active in view of cancer prevention. On the other hand, promotion extends over a long period of time, and interrupting this process is considered as an effective cancer preventive measure.

From the above-mentioned perspective, research on anti-carcinogenesis promoters reached the various reports on effects of various plant extracts and their ingredients as anti-carcinogenesis promoters. However, supply of harmless plant extracts originated from edible plant with higher anti-carcinogenesis effects is in demand.

In this context, original extracts from various plants such as Phellodendri Cortex and others are reported as possible carcinogenesis inhibitors on experimental basis, and the details are disclosed step by step such as that a β-carotene is an active ingredient.

Epstein-Barr virus genome inactivating test is carried out as the first-stage screening test of anti-carcinogenesis promoter. In this test, inhibition of virus genome incident with tetradecanoylphorbolacetate (hereinafter called “TPA”) as its promoter, in a culture system of Raji cells is used as an indicator. The mentioned Raji cells are culture cells originated in species of parakeet lymph and holds Epstein-Barr virus genome inside. This method is not only rapid and quantitative but also available for detection of active substances in a small quantity.

Epstein-Barr virus is a virus of the herpesvirus family and regarded as the cause of parakeet lymph and epipharynx. This virus is not detected from these cancer patients only but spreads around the world as a universal virus in human race and almost all adults are believed to be infected with the Epstein-Barr virus. It is evidenced that the Epstein-Barr virus makes Normal B lymphocyte of human its infectious target, goes bulbous, then this virus gives the unlimited reproduction capability to the B lymphocyte. This virus is defined as a common virus factor of human with primordial tumor toward B lymphocyte.

The object of this invention is to provide a new carcinogenesis inhibitor containing carcinogenetive effectiveness.

### DISCLOSURE OF THE INVENTION

The present inventors repeated a systematic study and experiments of various microbes and plants with Epstein-Barr virus genome inactivating as its indicator and reached this invention with a remarkable discovery of strong Epstein-Barr virus genome inactivating effects in extracts from paradicsom paprika and carotenoids contained in the said extracts such as capsanthin, fatty acid esters of capsanthin, capsorubin diester, capsanthin3,6-epoxide, capsorubin and cucurbitaxanthin A-3′ ester.

Besides, as a result of the study on a structure of capsanthin esters the inventor discovered a stronger Epstein-Barr virus genome inactivating effects in capsanthin monoester, particularly capsanthin. diester.

Moreover, as a result of detailed study on the Epstein-Barr virus genome inactivating effects in fatty acid ester of capsanthin, capsorubin and cucurbitaxanthin, it was discovered that palmitic acid esters, lauric acid esters and myristic acid esters have the carcinogenesis inhibitory effects.

The inventor selected a fruit of paradicsom paprika as a plant containing a large amount of the above-mentioned active ingredients. Paradicsom paprika is a vegetable originated in Hungary. Its original fruit is in clover shape, deep red with wax-type shiny surface and its color doesn't change by heating. Its fruit is thick, sugary and not grassy. The example of its ingredient analysis shows that the fruit contains 780 of vitamin A effect (IU), 0.23 mg of vitamin B2, 189 mg of vitamin C and 0.62 mg of iron per 100 g of edible part. In this invention, paradicsom paprika means the original species of paradicsom paprika, however, this definition also includes the species which are improved its certainty of crop by continuous selection and fixation of specific fruit of the original species (hereinafter called “Pure-line Species”). This Pure-line Species is classified as Capsicum annuum L. var. grossum in taxonomy. For instance, the species includes “Tomapi” (trademark) in the market of Japan. In addition to this Pure-line Species, this definition includes the species made by crossing the Pure-line Species and one selected from large bell-type, pimento-type, Hungarian paprika-type or large Neapolitan-type (hereinafter called “F1 Species”), and the species made by crossing plural species of F1 Species (hereinafter called “Four Element Crossing Species”) and restored species of these crosses (hereinafter called “F2 Species”). That is, in this invention, paradicsom paprika includes its original species, Pure-line Species, F1 Species, Four Element Crossing Species, F2 Species and crossing species of paradicsom paprika thereafter.

Active ingredients of carcinogenesis inhibitor of this invention such as carotenoids of capsanthin, fatty acid esters of capsanthin, capsorubin diester, capsanthin 3,6-epoxide, capsorubin and cucurbitaxanthin-A-3′-ester can be separated from fruit of the genus Capsicum such as a so-called red pepper. Specifically, paradicsom paprika contains a large amount of these carotenoids which can be separated and used effectively. In this invention, capsanthin, fatty acid esters of capsanthin, capsorubin diester, capsanthin 3,6-epoxide, capsorubin and cucurbitaxanthin-A-3′-ester which are contained in extract from paradicsom paprika by using acetone and the like are preferable. However, this is not limited to extracts from the above-mentioned species but other plants such as fruit of red pepper or synthetic substance has no problem to be used. Also, the extract method is not specified and different solvent for extraction may be used.

Moreover, palmitic acid, lauric acid, and myristic acid are discovered as fatty acid components of capsanthin esters, capsorubin diester and cucurbitaxanthin A-3′.

Capsanthin, fatty acid esters of capsanthin, capsorubin diester, capsanthin 3,6-epoxide, capsorubin and cucurbitaxanthin-A-3′-ester indicated strong carcinogenesis inhibitory effects. These carotenoids inactivate Epstein-Barr virus genome and produce carcinogenesis inhibitor effects. Accordingly, from this standpoint of cancer prevention, these carotenoids can be applied in drugs, cosmetics, health foods and other fields.

### BRIEF DESCRIPTION OF THE DRAWINGS

FIG. 1 is the graphic to show the relation between a lapse of weeks after the beginning of experiment and the ratio of mice with activated papilloma in the mouse skin under 2-stage carcinogenesis inhibitory examination using extracts of paradicsom paprika.

FIG. 2 is the graphic to show the relation between a lapse of weeks after the beginning of experiment and the number of activated papilloma in the mouse under skin 2-stage carcinogenesis inhibitory examination using extracts of paradicsom paprika.

FIG. 3 shows ¹H-NMR of cap sorubin diester.

FIG. 4 shows ¹³C-NMR of capsorubin diester.

FIG. 5 shows UV-VIS of capsorubin diester.

FIG. 6 shows FAB-MS of capsorubin diester.

FIG. 7 shows ¹H-NMR of capsorubin diester.

FIG. 8 shows ¹³C-NMR of capsorubin diester.

FIG. 9 shows UV-VIS of capsorubin diester.

FIG. 10 shows FAB-MS of capsorubin diester.

FIG. 11 shows ¹H-NMR of cucurbitaxanthin.

FIG. 12 shows UV-VIS of cucurbitaxanthin.

FIG. 13 shows FAB-MS of cucurbitaxanthin.

FIG. 14 shows ¹H-NMR of capsanthin 3,6-epoxide.

FIG. 15 shows ¹³C-NMR of capsanthin 3,6-epoxide.

FIG. 16 shows UV-VIS of capsanthin 3,6-epoxide.

FIG. 17 shows FAB-MS of capsanthin 3,6-epoxide.

FIG. 18 shows ¹H-NMR of cap sorubin.

FIG. 19 shows UV-VIS of capsorubin.

FIG. 20 shows FAB-MS of capsorubin.

FIG. 21 shows ¹H-NMR of cap santhin monoester.

FIG. 22 shows ¹³C-NMR of cap santhin monoester.

FIG. 23 shows UV-VIS of capsanthin monoester.

FIG. 24 shows FAB-MS of capsanthin monoester.

FIG. 25 shows ¹H-NMR of capsanthin.

FIG. 26 shows ¹³C-NMR of capsanthin.

FIG. 27 shows UV-VIS of capsanthin.

FIG. 28 shows FAB-MS of capsanthin.

FIG. 29 is the graphic to show the relation between a lapse of weeks after the beginning of experiment and the ratio of mice with activated papilloma in the mouse skin under 2-stage carcinogenesis inhibitory examination using capsanthin and the like.

FIG. 30 is the graphic to show the relation between a lapse of weeks after the beginning of experiment and the number of activated papilloma in the mouse skin under 2-stage carcinogenesis inhibitory examination using capsanthin and the like.

### THE BEST EMBODIMENTS TO CARRYING OUT THE INVENTION

### Procedure of Experiment 1

Extracts from paradicsom paprika of this invention are extracted under the following procedure. For this experiment example of extracts, the Pure-line Species described above (Species classified in botanical name: Capsicum annuum L. var. grossum) are used. In practice, 1 kg of edible parts of paradicsom paprika are cut off and immersed in acetone at room temperature in a dark place, kept stationary and shaken at times to extract red acetone extract liquid. With adding acetone into the remaining substance, the acetone extract liquid is collected three times in the same procedure. The acetone extracts are obtained by decompressing, concentrating, drying and making the extracted liquid extracts solid. In a different procedure, methanol extracts are obtained in the same method as above, using methanol in stead as solvent for extract. Then, the methanol extracts is dissolved in hexane and to obtain a hexane extracts liquid collecting the soluble parts of the mentioned methanol extracts. The hexane extracts are obtained by decompressing and concentrating the said collected soluble parts.

(Measurement of inactivation of Epstein-Barr virus genome incidence)

Measurement conditions of inactivation of Epstein-Barr virus genome incidence, using the acetone extracts and the hexane extracts, are as follows. PRMI1640 added embryonic serums and antibiotics are used as the culture liquid of Raji cells, virus latent infection-human lymphotropic bulbous cells. Under this culture conditions, the rate of natural incidence of Epstein-Barr virus of early antigens are not more than 0.1%. Raji cells, adjusted at the concentration of 1×10⁶ cell/ml is cultured for 48 hours at 37° C. in the above described culture liquid after adding 4 mM of n-butyric acid, 20 ng/ml of TPA and 100 μg/ml of test substance. Then cells which generated Epstein-Barr virus early antigens are detected in the indirect immunofluorescence method using serums from epipharynx cancer patients. The ratio of positive cells calculated against the control without adding testisubstance is regarded as inactivation of virus genome incidence. In addition, the same inactivation were measured as varying the concentration of the test substance to 10, μg/ml and 1 μg/ml. The results are shown in the table 1 below.

TABLE 1
Unit: % inhibitory rate (% survival rate of Raji cells)

| Concentration (μg/TPA) | 100 | 10 | 1 |
|---|---|---|---|
| Paprika acetone extract | 100(70) | 71.0(>80) | 8.2(>80) |
| Paprika hexane extract | 100(70) | 56.3(>80) | 0 (>80) |
| Phellodendri cortex extract | 100(70) | 72.4(>80) | 15.1(>80) |

As the table 1 shows, extracts from paradicsom paprika using acetone and extracts from the aforementioned extracts using hexane showed strong inactivate effect of carcinogenesis virus. Its inhibitory effect has almost the same effect of Phellodendri Cortex extracts, which are used as medical supplies. Survival rates of Raji cells are sustained at least 70% and no particular virulence against cells was observed. Accordingly, extracts from paradicsom paprika were proved to have the carcinogenesis inhibitory effect and can be used as active ingredients of carcinogenesis inhibitor.

(two-stage carcinogenesis inhibitory test on mouse skin)

As mentioned above, it is confirmed that the extracts from paradicsom paprika shows strong inactivate effect against carcinogenesis virus. Now, skin cancer inhibitory effects were tested on mice to define its carcinogenesis inhibitory effects. Conditions of this two-stage carcinogenesis inhibitory test on mouse skin were as follows:

Body hair of one group of 15 ICR female mice (age 6 weeks) on their back were shaved and after 24 hours, applied (100 μg, 390 nmol) of 7,12-dimethylbenz[α]anthracene (hereinafter called “DMBA”) dissolved in acetone (0.1 ml) on their shaved skin of back as an initiation. Since one week later, each group of mice had been treated as follows:

First group: TPA (1 μg, 1.7 nmol) dissolved in acetone (0.1 ml) had been applied for 20 consecutive weeks, twice a week, as a promotion. During this treatment, additional acetone (0.1 ml) was applied on the same part one hour prior to each TPA application. (positive control group).

Second group: As the first group, TPA (1 μg, 1.7 nmol) dissolved in acetone (0.1 ml) had been applied for 20 consecutive weeks, twice a week, as a promotion but 50 μg of paradicsom paprika extracts (extracted by methanol), the subject material, dissolved in acetone (0.1 ml) was applied one hour prior to each application instead of additional acetone.

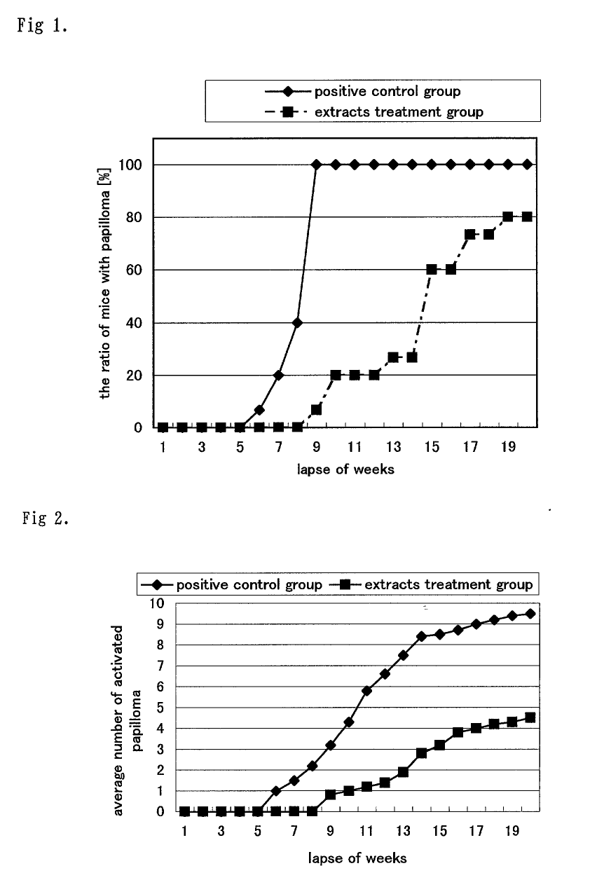

Until the 20^{th} week since the start of a promotion by TPA application, incidence of papilloma on back of mice had been observed once a week. And the rate of mice with papilloma incidence and the average number of papilloma incidence per mouse were evaluated based on the comparison of the positive control group and the second group. The results are shown in FIG. 1 and 2.

As the results shown in FIG. 1, the first tumor was formed on the 7^{th} week after the start of the promotion and by the 11^{th} week, tumors were found on all of the mice in the positive control group. In the second group (methanol extracts treatment group), the first tumor was formed on the 9^{th} week which proved its effects to delay tumor formation. Moreover, 20% of mice in the second group was still free from tumor formation at the end of this 20-week experiment.

Further, as FIG. 2 shows, the average number of tumors per mouse in the positive control group after 20 weeks was 9.1 but was 4.5 in the second group which proved its 50% of carcinogenesis inhibitory effects.

From the above-mentioned results, the extracts from paradicsom paprika were proved to contain carcinogenesis inhibitory effects in the two-stage carcinogenesis inhibitory test on mouse skin.

### Procedure of Experiment 2

As mentioned above, extracts from paradicsom paprika are proved in medical experiments to act as carcinogenesis inhibitor and have an inactive effect of carcinogenesis virus. Thereupon, the present inventors moved on to the following tests to prove active ingredients contained in the extracts.

Carotenoids, an active ingredient of carcinogenesis inhibitor of this invention, are for example extracted and separated from paradicsom paprika as follows.

For this experiment example of extracts, the Pure-line Species described above (Species classified in botanical name: Capsicum annuum L. var. grossum) are used. In practice, 800 g of edible parts of paradicsom paprika are cut off and immersed in acetone at room temperature in dark place, kept stationary and shaken at times to extract red acetone extracts. With adding acetone into the remaining substance, the acetone extract liquid is collected three times in the same procedure. The acetone extract liquid is obtained by decompressing, concentrating, drying and making the extracted liquid extracts solid. In a different procedure, methanol extracts are obtained in the same method as above, using methanol in stead as solvent for extract. Then, the methanol extracts is dissolved in hexane and to obtain a hexane extracts liquid collecting the soluble parts of the mentioned methanol extracts. The hexane extracts are obtained by decompressing and concentrating the said collected soluble parts.

Based on quantitative analysis of the extracts, they proved that 120 g of carotenoids could be extracted from 800 g of edible parts of paradicsom paprika. In order to analyze ingredients of these carotenoids, the hexane extracts were applied to column-chromatography, using silica gel as its adsorbent. And 20 mg of Ingredient A (capsorubin diester) was separated from fraction eluted by hexane-ether (7:3) as well as 40 mg of Ingredient B (capsanthin diester) from fraction eluted by hexane-ether (8:2), 8 mg of Ingredient C (cucurbitaxanthin) from fraction eluted by ether (9:1), 8 mg of Ingredient D (capsanthin epoxide) from fraction eluted by ether-acetone (9:1), 5 mg of Ingredient E (capsorubin) from fraction eluted by ether-acetone (7:3), 24 mg of Ingredient F (capsanthin monoester) from fraction eluted by hexane-ether (5:5) and 12 mg of Ingredient G (capsanthin) from fraction eluted by ether-acetone (8:2).

In order to specify separated Ingredients A to G mentioned above, each substance of each ingredient was analyzed by using Ultraviolet-visible Absorption Spectrum (hereinafter called “UV-VIS”), Fast Atom Bombardment Mass Spectrometry Spectrum (hereinafter called “FAB-MS), Proton Magnetic Resonance Spectrum (hereinafter called “¹H-NMR) or Carbon-13 Magnetic Resonance Spectrum (hereinafter called “¹³C-NMR) as required.

(Analysis of Ingredient A)

Based on the above analysis, Ingredient A was proved to be capsorubin diester. In the procedure described above, this ingredient was extracted from paradicsom paprika extracts and the kinds of its fatty acid and the component ratio were proved as shown in table 2. The spectrum data is as follows:

UV-VIS (ether)λ:445,479,510,nm, 1H-NMR (CDCl₃) δ:0.86 (6H, s, H-16, 16′), 0.88 (6H, t, J=7 Hz, CH₃ fatty acid), 1.18 (6H, s, H-17, 17′), 1.25 (s, CH₂ fatty acid), 1.32 (6H, s, H-18, 18′), 1.57 (2H, dd, J=15, 3.5 Hz, H-4β, 4′β), 1.74 (2H, dd, J=13.5, 4 Hz, H-2 α, 2′β), 1.96 (6H, s, H-19, 19′), 1.99 (6H, s, H-20, 20′), 2.09 (2H, dd, J=13.5, 8Hz, H-2 α, 2′α), 2.27 (4H, t, 7Hz, CH₂ fatty acid), 2.99 (2H, dd, J=15, 9 Hz, H-4 α, 4′α), 5.24 (2H, m, H-3, 3′), 6.36 (2H, m, H-14, 14′), 6.44 (2H, d, J=15 Hz, H-7, 7′), 6.55 (2 H, d, J=15 Hz, H-12, 12′),6.59 (2H, d, J=11 Hz, H-10, 10′), 6.68 (2H, dd, J=15, 11 Hz, H-11, 11′), 6.70 (2H, m, H-15, 15′), 7.34 (2H, d, J=15 Hz, H-8, 8′), ¹³C-NMR (CDC₁₃) δ: 12.80 (C-20, 20′), 12.87 (C-19, 19′), 14.13 (CH₃ fatty acid), 20.78 (C-18, 18′), 22.69 (CH₂ fatty acid), 24.77 (CH₂ fatty acid), 25.05 (C-17, 17′), 25.61 (C-16, 16′), 29.14, 29.27, 29.35, 29.47, 29.60, 29.64, 31.91, 34.65 (CH₂ fatty acid), 42.20 (C-4, 4′), 43.73 (C-1, 1′), 47.66 (C- 2, 2′), 58. 51 (C-5, 5′), 73.24 (C-3, 3′), 120.80 (C-7, 7′), 124.60 (C-11, 11′), 131.21 (C-15, 15′), 133.95 (C-9, 9′), 134.99 (C-14, 14′), 136.94 (C-13, 13′), 140.70 (C-10, 10′), 141.82 (C-12, 12′), 147.02 (C-8, 8′), 173.63 (C=O fatty acid), 202.51 (C-6, 6′), FAB-MS m/z: 1076 (M⁺) for C₇₂H₁₁₆O₆ (capsorubin-dipalmitate), 104 8 (M⁺) for C ₇₀H₁₁₂O₆ (capsorubin-palmitate, myristate), 10 20 (M⁺) for C₆₈H ₁₀₈O ₆ (capsorubin-dimyristate), 992 (M^{+) for C}₆₆H_{104 O}₆ (capsorubin-myristate, laurate), 964 (M⁺) for C₆₄H ₁₀₀O₆ (capsorubin-dilaurate), the ester component ratio of capsorubin-diester fatty acid dipalmitate:palmitate, myristate:dimyristate:myristate, laurate:dilaurate (6:18:41:24:11)

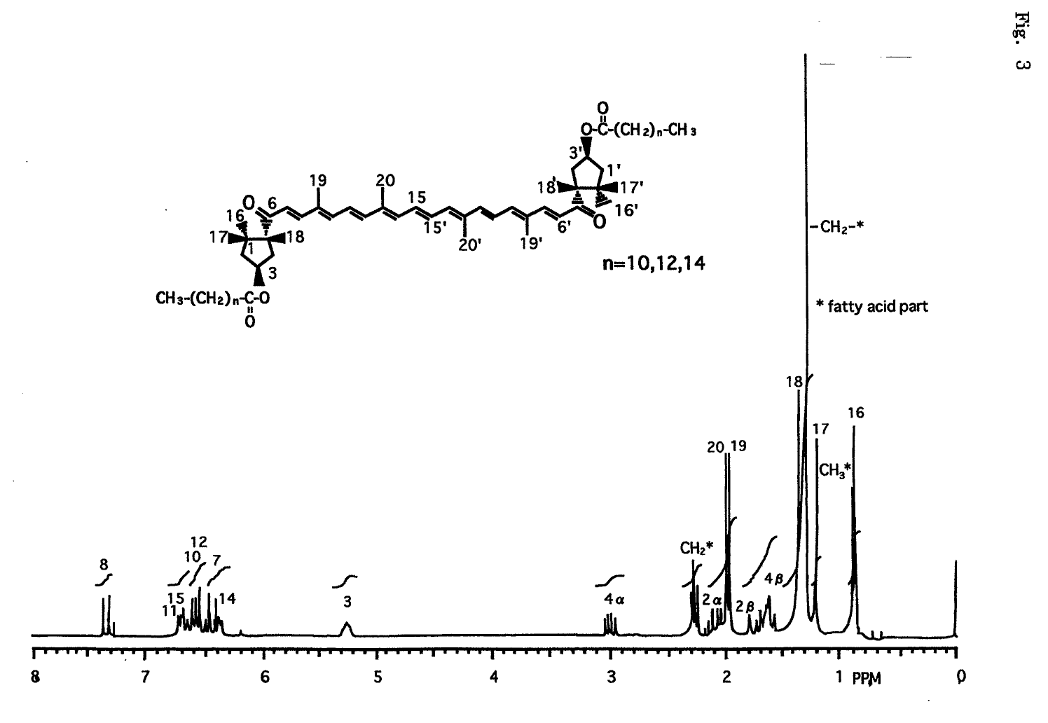

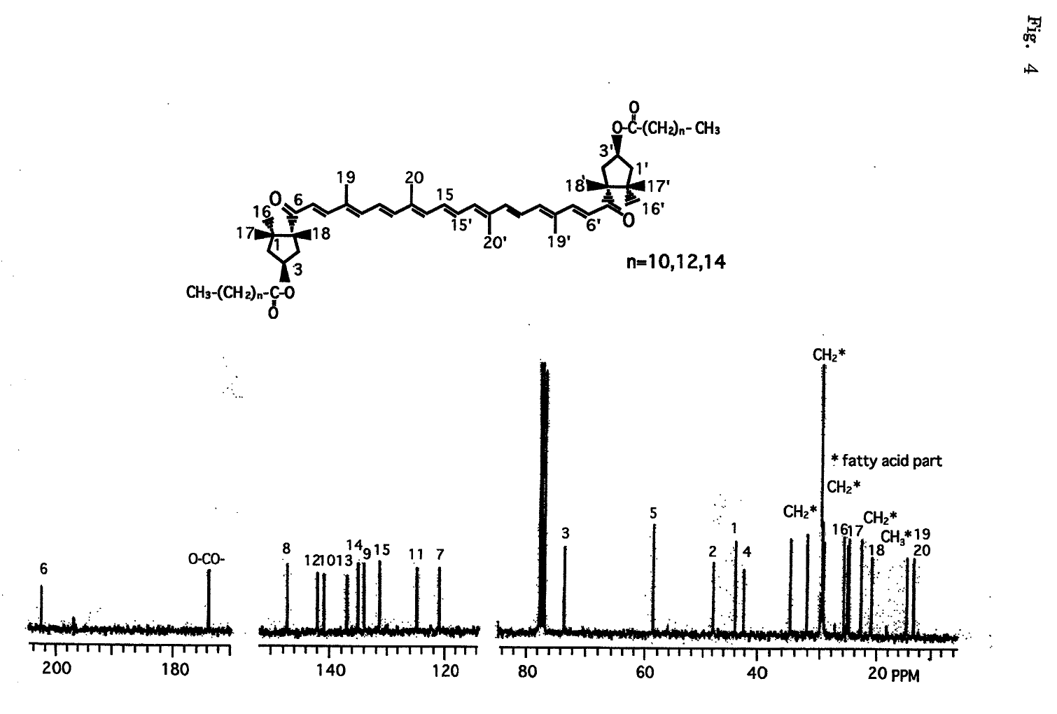

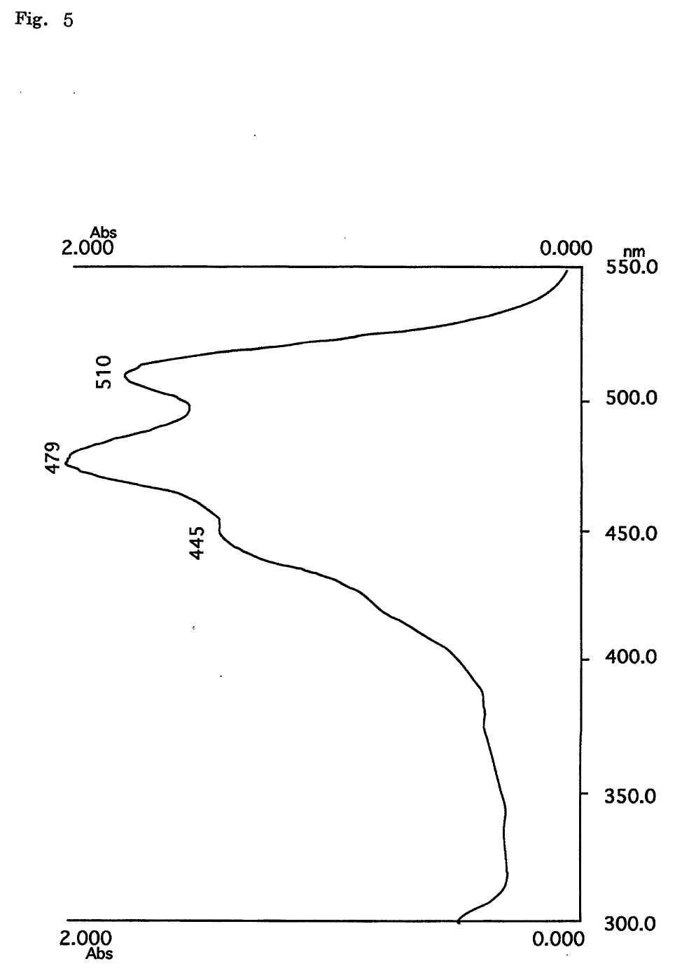

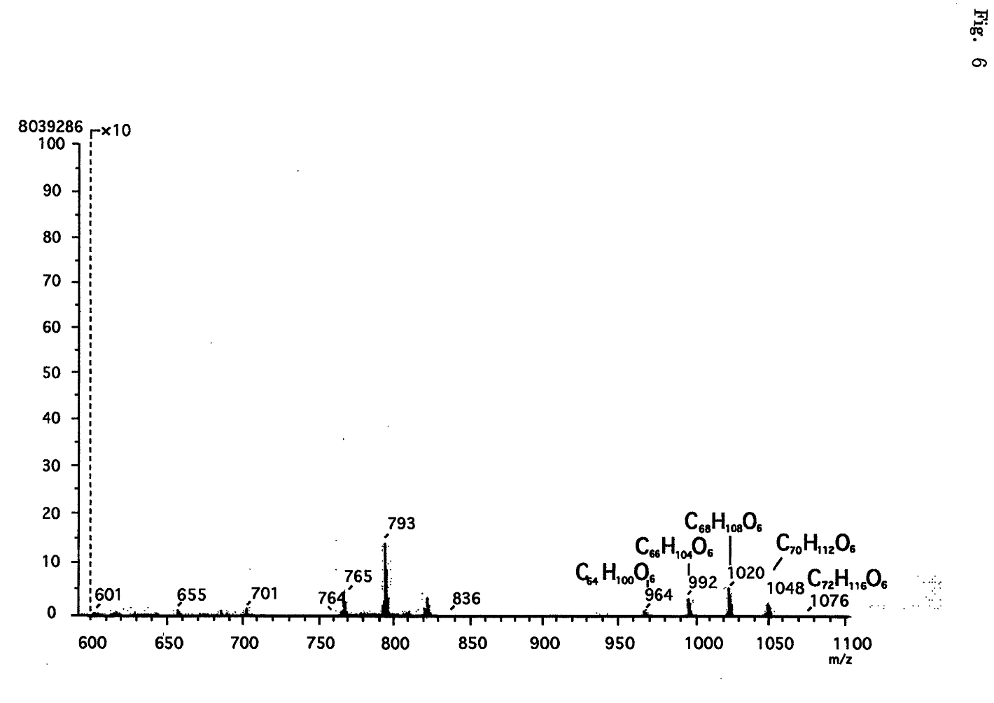

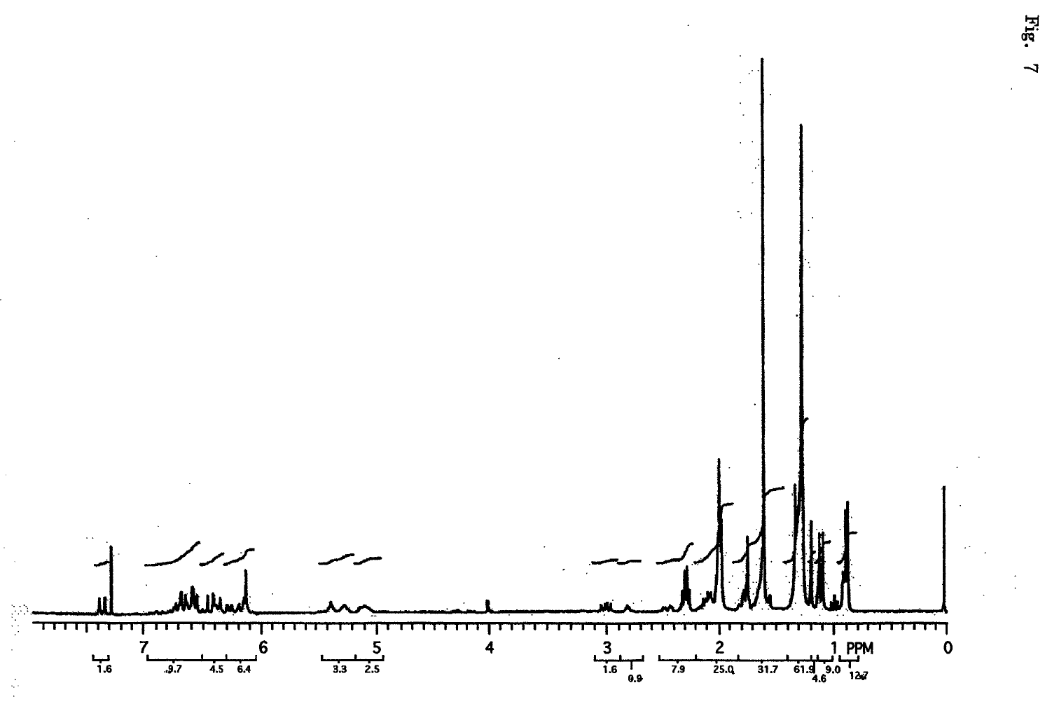

FIG. 3 is the chart of ¹H-NMR of capsorubin-diester, and FIG. 4 is the chart of ¹³C-NMR. Further, FIG. 5 is the chart of UV-VIS and FIG. 6 is the FAB-MS chart of capsorubin-diester.

TABLE 2

| | Molecular | |
|---|---|---|
| Component of Fatty Acid | weight | Component Ratio |
| Palmitic, Palmitic | 1076 | 6% |
| Myristic, Palmitic | 1048 | 18% |
| Myristic, Myristic | 1020 | 41% |
| Lauric,Myristic | 992 | 24% |
| Lauric, Lauric | 964 | 11% |

(Analysis of Ingredient B)

Based on the above-mentioned analysis, Ingredient B was proved to be capsanthin diester. The kinds of its fatty acid and the component ratio in this procedure were proved as shown in table 3. Its spectrum data is as follows:

UV-VIS (ether) λ:468, 496 nm, ¹H-NMR (CDCl₃) δ:0.86 (3H, s, H-16′), 0.88 (6H, t, J=7 Hz, CH₃ fatty acid), 1.08 (3H, s, H-16), 1.11 (3H, s, H-17), 1.18 (3H, s, H-17′), 1.25 (s, CH₂ fatty acid), 1.32 (6H, s, H-18′), 1.58 (1H, dd, J=12, 12 Hz, 2β), 1.57 (1H, dd, J=15, 3.5 Hz, H-4′β), 1.78 (1H, dd, J=13.5, 4 Hz, H-2′β), 1.72 (3H, s, H-18), 1.77 (1H, ddd, J=12, 4, 1.5 Hz, H-2α), 1.96 (3H, s, H-19′) 1.97 (6H, s, H-19, 20), 1.99 (3H, s, H-20′), 2.09 (1H, dd, J-13.5, 8 Hz, H-2′α), 2.11 (1H, dd, J=15.5, 11 Hz, H-4 β), 2.45 (1H, ddd, J=15.5, 5.5, 1, 5 Hz, H-4α), 2.27 (4H, t, 7 Hz, CH₂ fatty acid), 2.99 (1H, dd, J=15, 9 Hz, H-4′α), 5.06 (1H, m, H-3), 5.24 (1H, m, H-3, 3′), 6.13 (2H, d, AB -type, H-7, 8), 6.16 (1H, d, J=11 Hz, H-10), 6.23 (1H, d, J=10.5, H-14), 6.36 (1H, d, J=15 Hz, H-14′), 6.36 (1H, d, J=11 Hz, H-14′), 6.44 (1H, d, J=15Hz, H-7′), 6.55 (1H, d, J=15 Hz, H-12′), 6.59 (1H, d, J=11 Hz, H-10′), 6.64 (1H, dd, J=15, 11 Hz, H-11), 6.68 (1H, dd, J=15, 11 Hz, H-1 1′), 6.70 (2H, m, H-15, 15′), 7.34 (1H, d, J=15 Hz, H-8′), ¹³C-NMR (CDCl₃) δ: 12.74 (C-19, 20), 12.80 (C-20′), 12.87 (C-19′), 14.13 (CH₃ fatty acid), 20.78 (C-18′), 21.53 (C-18), 22.69 (CH₂ fatty acid), 24.77 (CH₂ fatty acid), 25.05 (C-17′), 25.51 (C-16′), 28.62 (C-16), 29.14, 29.27, 29.35, 29.47, 29.60, 29.64(CH₂ fatty acid), 30.16 (C-17), 31.91, 34.65 (CH₂ fatty acid), 36.82 (C-1) 38.60 (C-4), 42.20 (C-4′), 43.73 (C-1′), 44.11 (C-2), 47.66 (C-2′),58.51 (C-5′), 68.50 (C-3), 73.24 (C-3), 120.80 (C-7′), 124.05 (C-11′), 125.51 (C-7), 127.84 (C-5), 124.60 (C-11′), 131.20 (C-10), 131.21 (C-15′), 132.35 (C-13), 133.95 (C-9′),134.99 (C-14′), 135.87 (C-9), 136.11 (C-14), 136.94 (C-13′), 137.60 (C-12), 137.71 (C-6), 138.41 (C-8), 140.70 (C-10′), 141.82 (C-12′), 147.02 (C-8′), 173.63 (C=O fatty acid), 202.51 (C-6′),FAB-MS m/z :1060 (M⁺) for C₇₂H₁₁₆O₅ (capsorubin-dipalmitate), 1032 (M⁺) for C₇₀H₁₁₂O₅ (capsorubin-palmitate, myristate), 1004 (M⁺) for C₆₈H₁₀₈O₅ (capsorubin-dimyristate), 976 (M⁺) for C₆₆H₁₀₄ O₅ (capsorubin-myristate, laurate), 948 (M⁺)for C₆₄ H¹⁰⁰O₅ (capsorubin-dilaurate), 920 (M⁺) for C₆₂ H₉₆O₅ (capsorubin-laurate, caprate), the fatty acid ester component ratio of capsorubin-diester; dipalmitate: palmitate, myristate: dimyristate: myristate, laurate dilaurate: laurate, caprate (4:14:35:36:10:1)

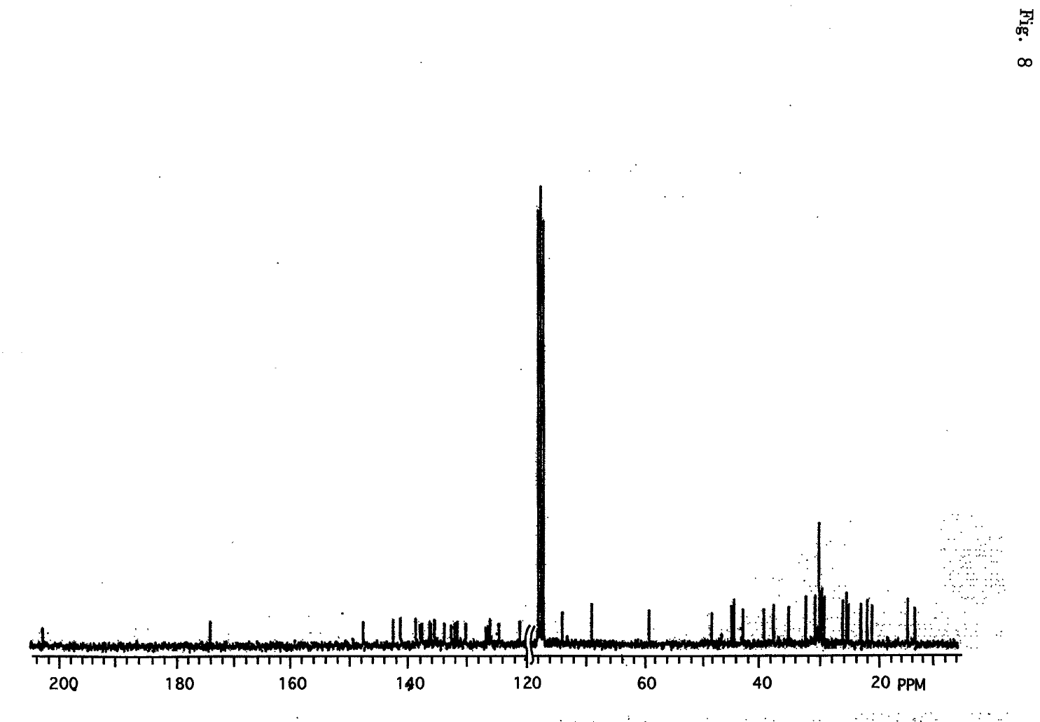

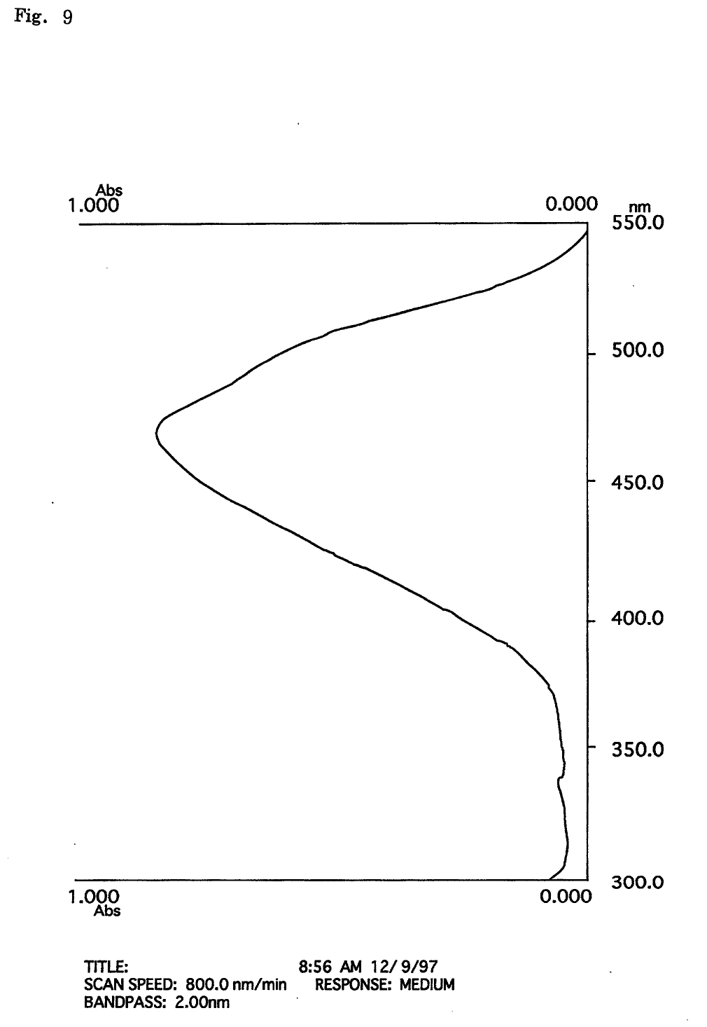

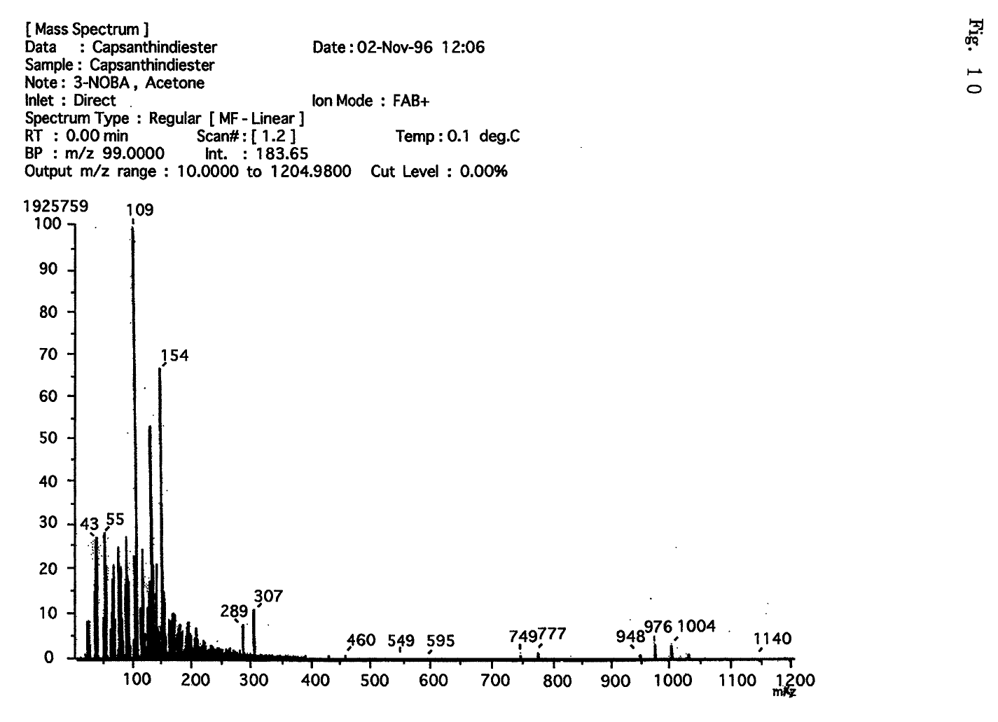

FIG. 7 is the chart of ¹H-NMR of capsorubin-diester, and FIG. 8 is the chart of ¹³C-NMR. Further, FIG. 9 is the chart of UV-VIS and FIG. 10 is the chart of FAB-MS of capsorubin-diester.

TABLE 3

| | Molecular | |
|---|---|---|
| Component of Fatty Acid | weight | Component Ratio |
| Palmitic, Palmitic | 1060 | 4% |
| Myristic, Palmitic | 1032 | 14% |
| Myristic, Myristic | 1004 | 35% |
| Palmitic, Lauric | 1004 | |
| Lauric, Myristic | 976 | 36% |
| Lauric, Lauric | 948 | 10% |
| Lauric, Capric | 920 | 1% |

(Analysis of Ingredient C)

Based on the above-mentioned analysis, Ingredient C was proved to be cucurbitaxanthin A-3′ esters. The kinds of its fatty acid and the component ratio in this procedure were proved as shown in table 4. Its spectrum data is as follows:

UV-VIS (ether) λ:425, 444, 472 nm, ¹H-NMR (CDCl₃) δ:0.88 (3H, s, H-17), 0.88 (3H, t, J=7 Hz, CH₃ fatty acid), 1.08 (3H, s, H-16′), 1.11 (3H, s, H-17′), 1.21 (3H, H-18), 1.25 (s, CH₂ fatty acid), 1.44 (3H, s, H-16), 1.61 (1H, d, J=11.5 Hz, H-2β), 1.72 (3H, s, H-18′), ˜1.84 (2H, m, H-2α, 2′- ax), 1.95 (3H, s, H-19), 1.97 (9H, s, H-20, 19′, 20′), 2.29 (2H, t, J=7, CH₂ fatty acid), 2.44 (1H, dd, J=16, 6 Hz, H-4′- eq), 4.40 (1H, t-like, J=7 Hz, H-3), 5.07 (1H, m, H-3′), 5.74 (1H, d, J=16 Hz, H-7), 6.10 (2H, m, H-7′, 8′), 6.16 (1H, d, J=11 Hz, H-10′), 6.20 (1H, d, J=11 Hz, H-10). 6.25 (2H, m, H-14, 14′), 6.36 (2H, d, J=15, H-12, 12′), 6.37 (1H, d, J=16 Hz, H-8), ˜6.62 (4H, m, H-11, 11′, 15, 15′), FAB-MS m /z 822 (M⁺) for C₅₆H₈₆O₄ (cucurbitaxathin-A-3′-palmitate), 794 (M⁺) for C₅₄H₈₂O₄ (cucurbitaxathin-A-3′-myristate), 766 (M⁺) for C₅₂H₇₈O₄ (cucurbitaxathin-A-3′-laurate), the fatty acid ester component ratio of cucurbitaxathin-A-esters; palmitate:myristate:laurate (20:57:23)

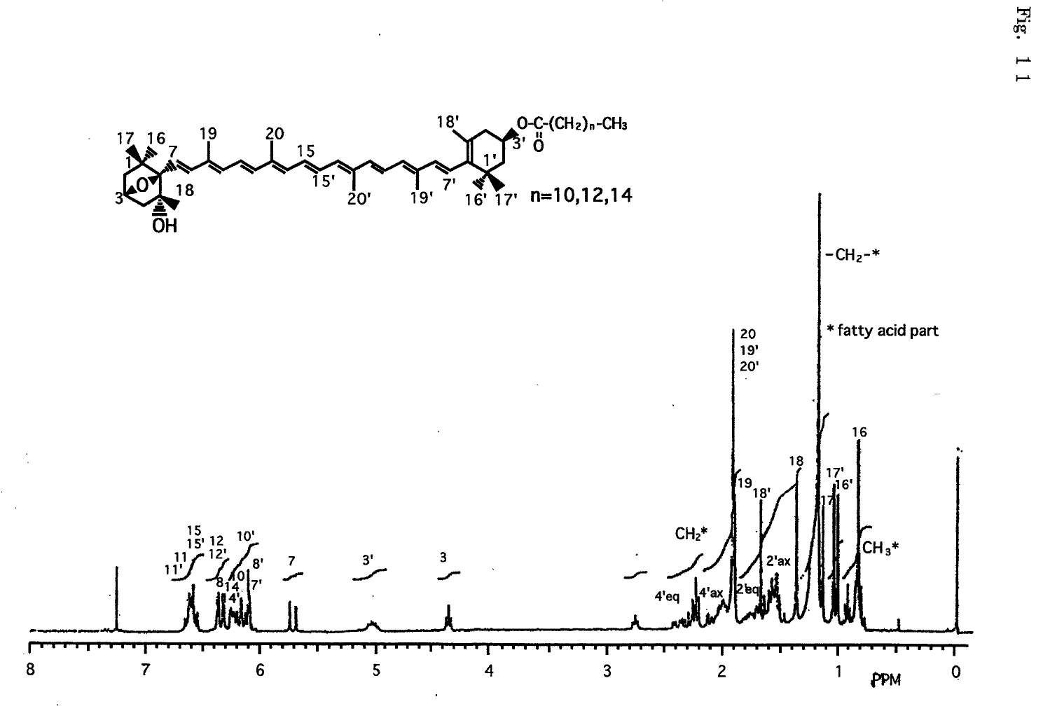

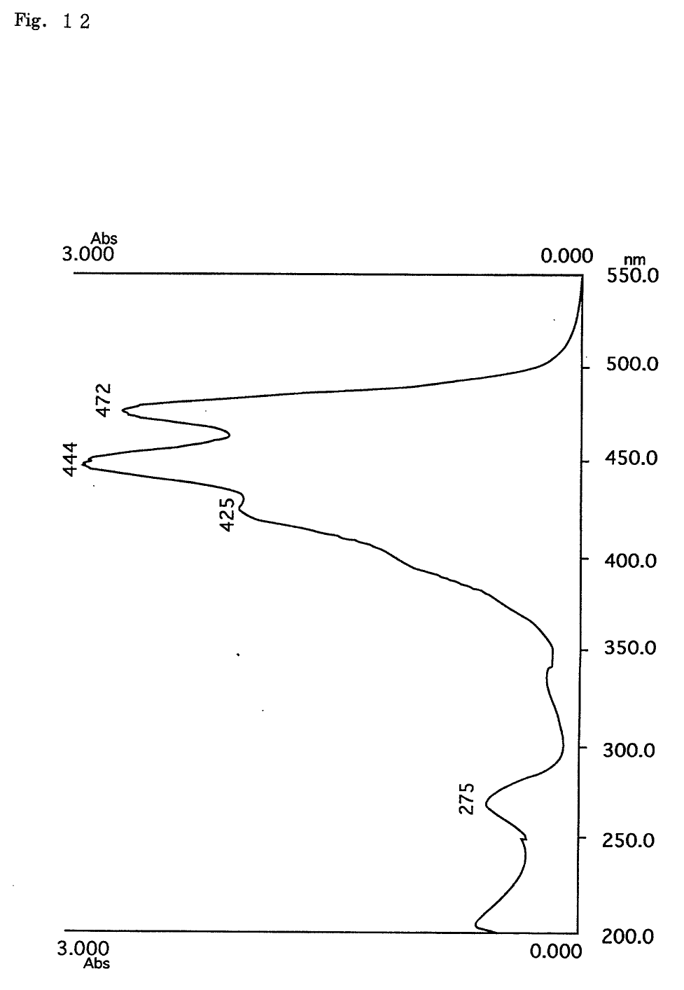

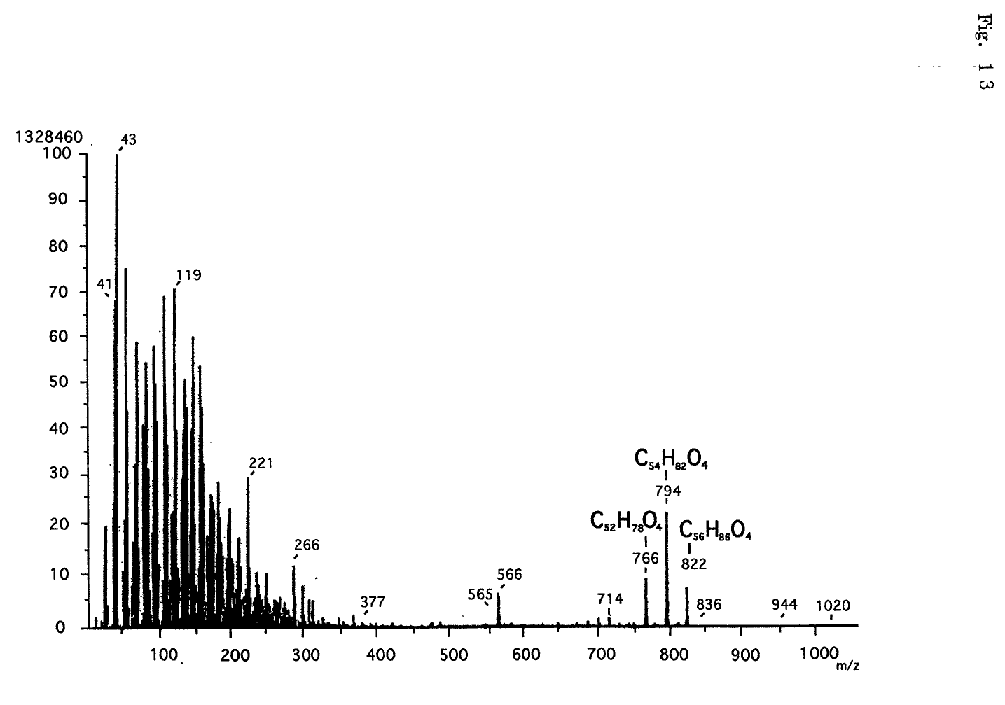

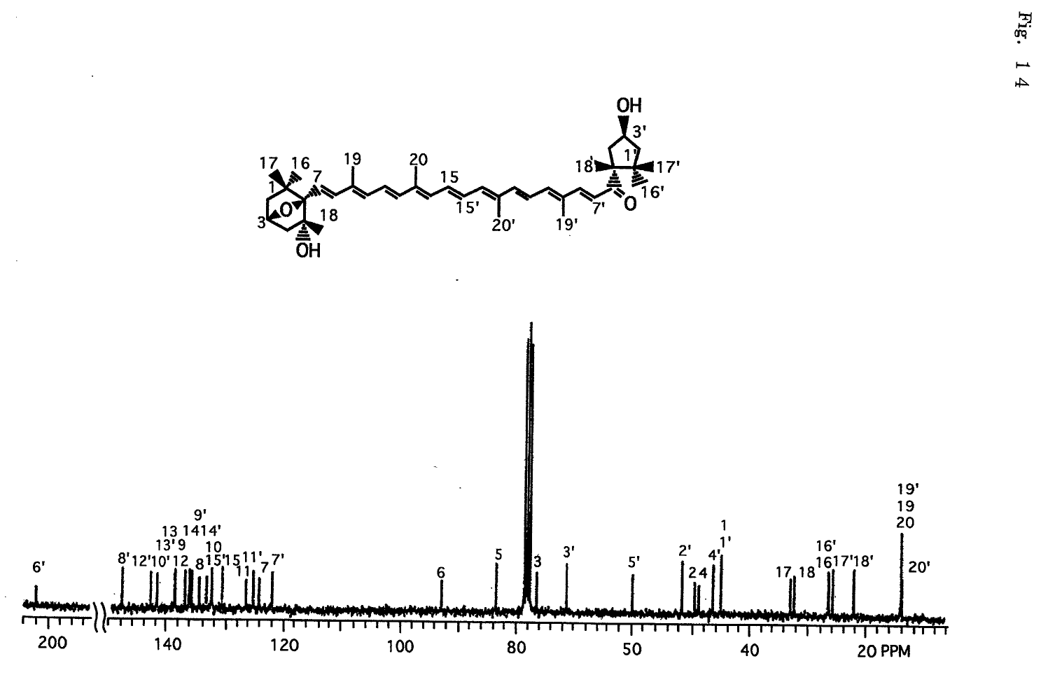

FIG. 11 is the chart of ¹H-NMR of cucurbitaxathin A-3′ esters, and FIG. 12 is the chart of UV-VIS. Further, FIG. 13 is the chart of FAB-MS.

TABLE 4

| | Molecular | |
|---|---|---|
| Component of Fatty Acid | weight | Component Ratio |
| Palmitic | 822 | 20% |
| Myristic | 794 | 57% |
| Lauric | 766 | 23% |

(Analysis of Ingredient D)

Based on the above-mentioned analysis, Ingredient D was proved to be capsanthin 3,6-epoxide. Its spectrum data is as follows:

UV-VIS (ether) λ:468 nm, ¹H-NMR (CDCl₃) δ:0.84 (3H, s, H-16′),0.88 (3H, s, H-1 7), 1.20 (3H, s, H-17′), 1.21 (3H, H-18), 1.37 (3H, s, H-18′), 1.44 (3H, s, H-16), 1.49 (1H, dd, J=15, 3.5 Hz, H-4′β), 1.61 (1H, d, J=11.5 Hz, H-2β), 1.97 (d, J=11.5 Hz, 4β), 1.71 (1H, dd, J=13.5, 4 Hz, H-2′β), 1.84 (ddd, J=11.5, 7, 2 Hz, H-2α), 1.96 (6H, s, H-19, 19′), 1.98 (6H, s, H-20, 20′), 2.00 (1H, dd, J=13.5, 8 Hz, H-2′α), 2.04 (1H, ddd, J=12, 7, 2, H-4α), 2.96 (1H, dd, J=15, 9 Hz, H-4′α), 4.40 (1 H, t-like, J=7 Hz, H-3), 4.51 (1H, m, H-3′), 5.76 (1H, d, J=16 Hz, H-7), 6.20 (1H, d, J=1 Hz, H-10), 6.70 (1H, d, J=11 Hz, H-14), 6.35 (1H, d, J=11 Hz, H-14′), 6.36 (1H, d, J=15 Hz, H-12), 6.38 (1H, d, J=16 Hz, H-8), 6.44(1 H, d, J=15 Hz, H-7′), 6.51 (1H, d, J=15 Hz, H -12′), 6.59 (1H, d, J=11 Hz, H-10′), ˜6.66 (4 H, m, H-11, 11′, 15, 15′), 7.34 (1H, d, J=15 Hz, H-8′), ¹³C-NMR (CDCl₃) δ:12.75 (C-20′), 12.88 (C-19, 20, 19′), 21.29 (C-18 ′), 25.09 (C-17′), 25.73 (C-16), 25.86 (C-16′), 31.58 (C-18), 32.16 (C-17), 43.97 (C-1,1′), 45.29 (C-4′), 47.71 (C-4), 48.49 (C-2), 50.83 (C-2′), 58.93 (C-5′), 70.34 (C-3′), 75.38 (C-3), 82.45 (C-5), 91.65 (C-6), 120.86 (C-7′), 123.11 (C-7), 124.08 (C- 11′), 125.40 (C-11′), 129.72 (C-15′), 131.60 (C-10), 132.44 (C-14), 133.62 (C-9′), 134.81 (C-8), 135.19 (C-9), 135.24 (C-14′),135.92 (C-13), 137.51 (C-13′), 135.92 (C-12), 140.71 (C-10′), 141.97 (C-12′), 146.87 (C-8′),202.93 (C-6′), FAB-MS m/z: 600 (M⁺) for C₄₀ H₅₆O₄

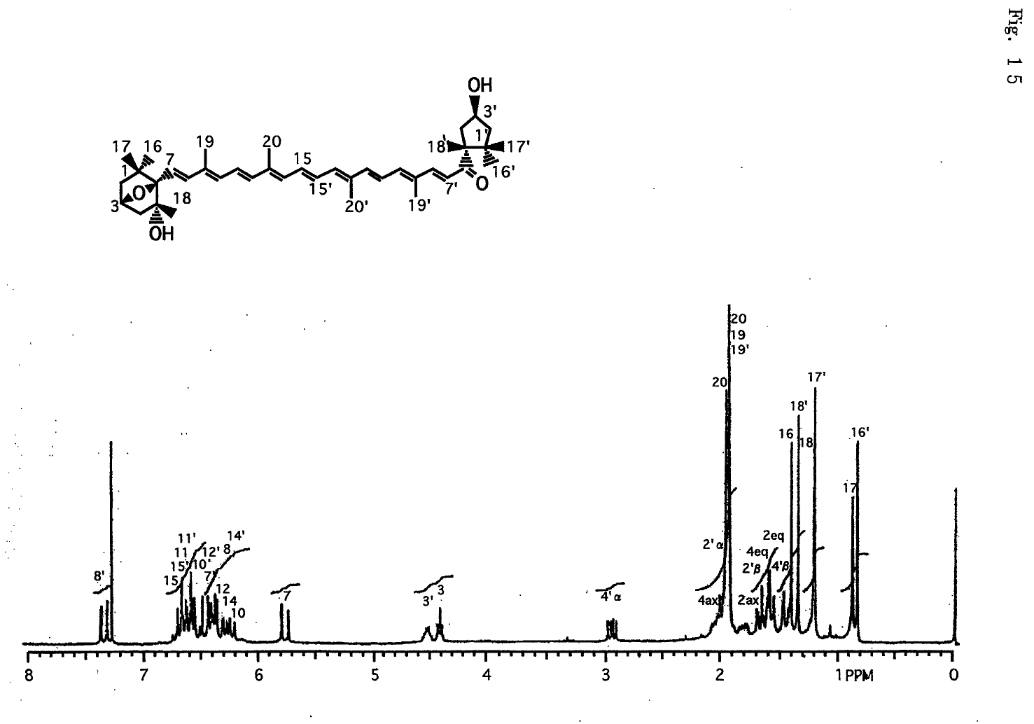

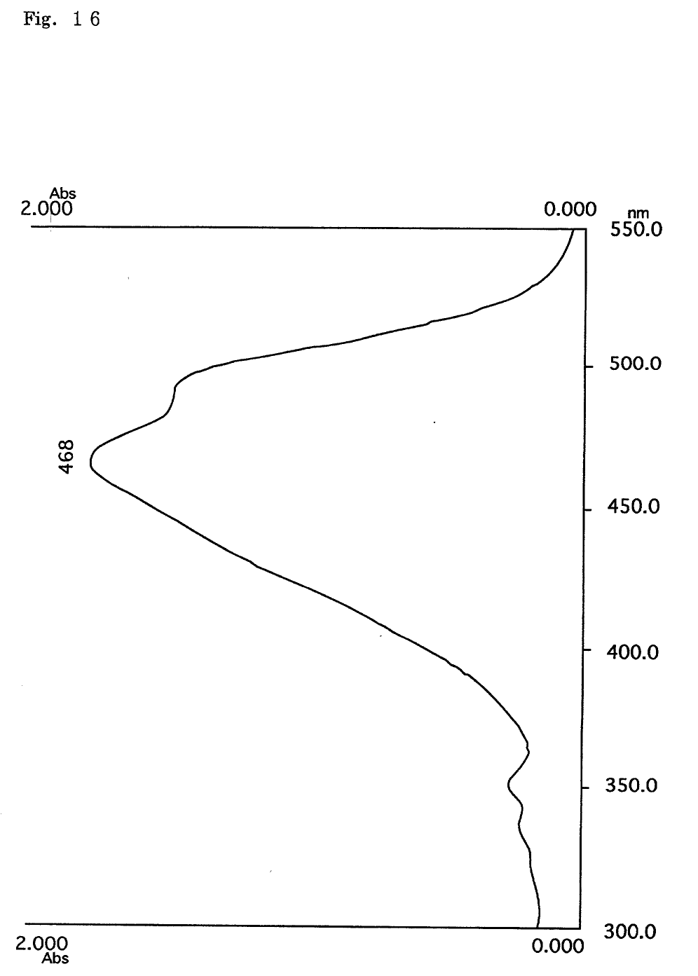

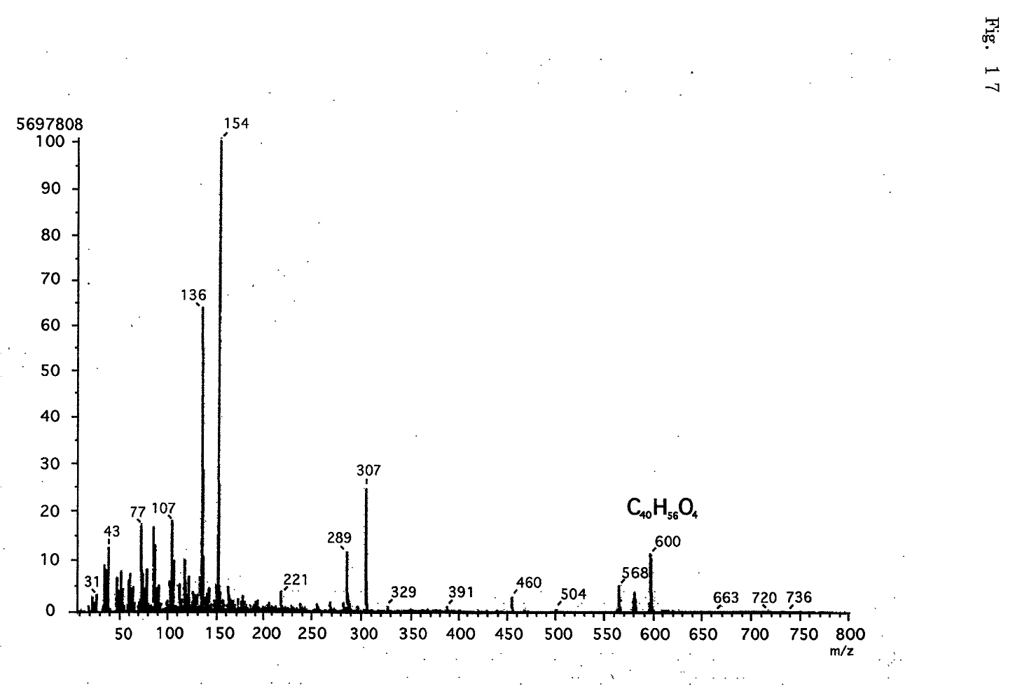

FIG. 14 is the chart of ¹H-NMR of capsanthin 3,6-epoxide and FIG. 15 is the chart of ¹³C-NMR. Further, FIG. 16 is the chart of UV-VIS and FIG. 17 is the chart of FAB-MS.

(Analysis of Ingredient E)

Based on the above-mentioned analysis, Ingredient E was proved to be capsorubin. Its spectrum data is as follows:

UV-VIS (ether) λ:445, 479, 510 nm, ¹H-NMR (CDCl₃) δ:0.84 (6H, s, H-16, 16′), 1.21 (6H, s, H-17, 17′), 1.37 (6H, s, H-18, 18′), 1.49 (2H, dd, J=15, 3.5 Hz, H-4 β, 4′β), 1.71 (2 H, dd, J=13.5, 4 Hz, H-2 β, 2′β), 1.96 (6H, s, H-19, 19′), 1.99 (6H, s, H-20, 20′), 2.00 (2 H, dd, J=13.5, 8 Hz, H-2α, 2′α), 2.96 (2H, d d, J=15, 9 Hz, H-4α, 4′α), 4.51 (2H, m, H-3, 3′), 6.36 (2H, m, H-14, 14′), 6.44 (2H, d, J=15 Hz, H-7, 7′), 6.55 (2H, d, J=15 Hz, H-12, 12′), 6.59 (2H, d, J=11 Hz, H-10, 10′), 6.68 (2H, dd, J=15, 11 Hz, H-11, 11′), 6.70 (2H, m, H-15, 15′), 7.34 (2H, d, J=15 Hz, H-8, 8′), FAB-MS m/z: 600 (M⁺) for C₄₀H₅₆O₄

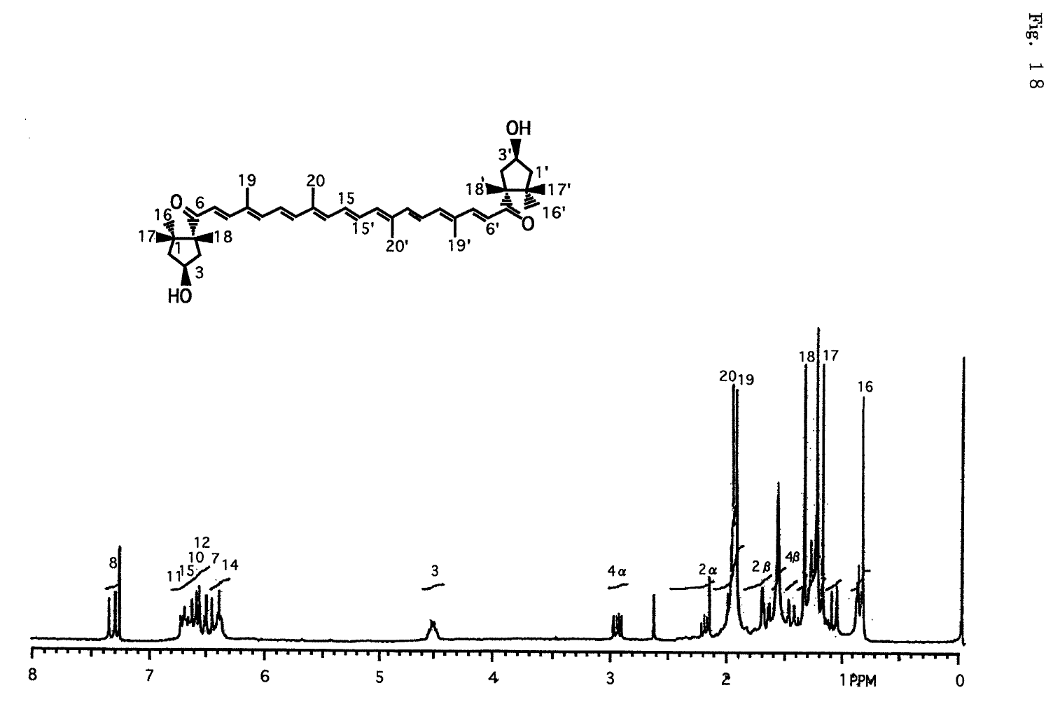

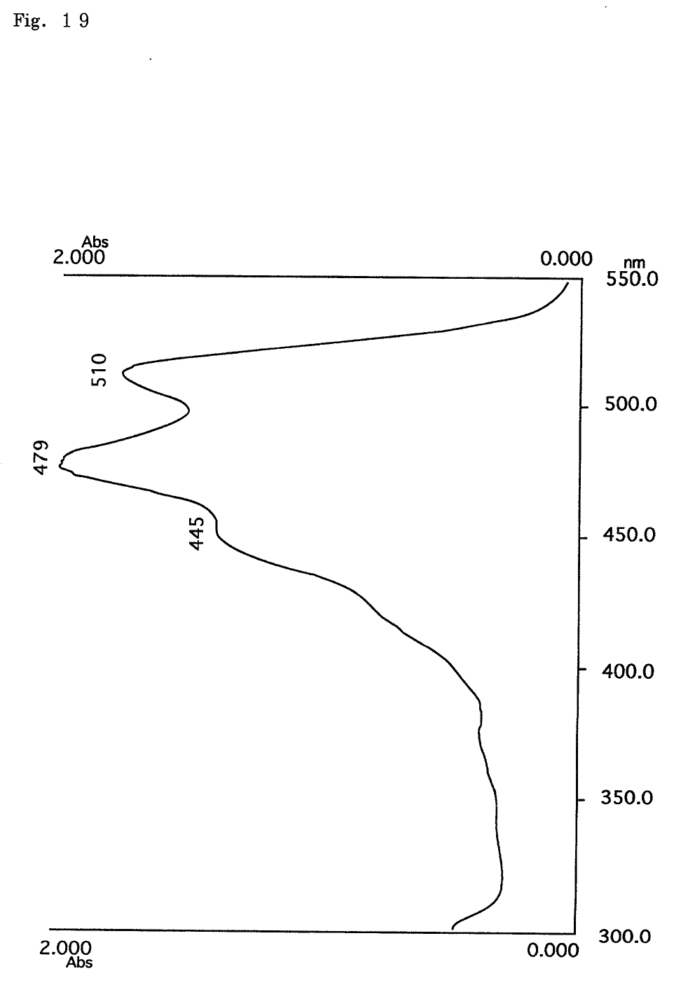

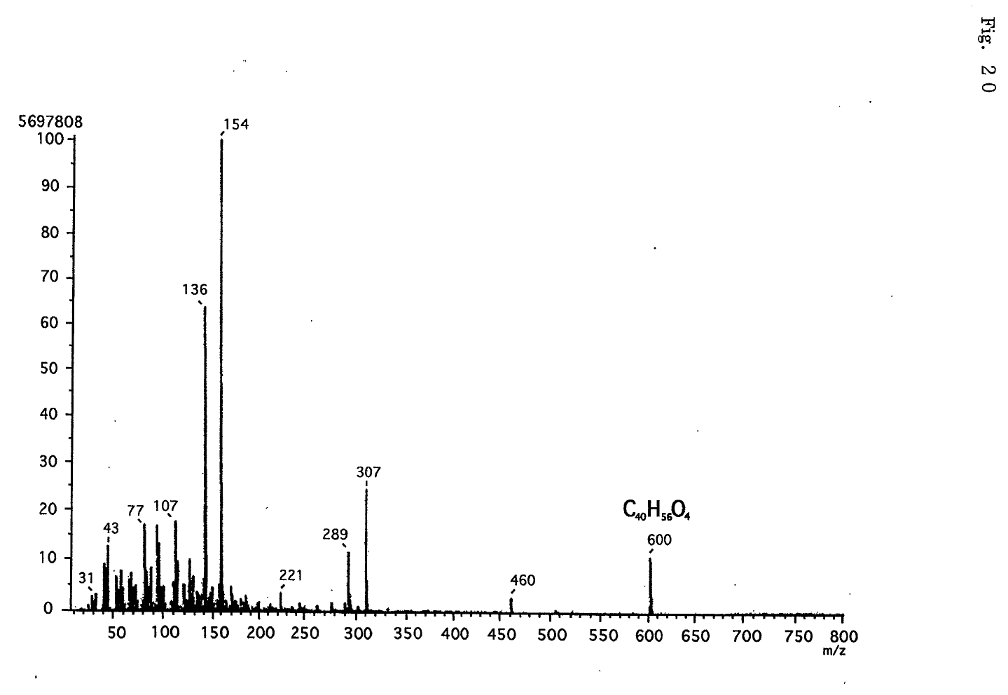

FIG. 18 is the chart of ¹H-NMR of capsorubin and FIG. 19 is the chart of UV-VIS. Further, FIG. 20 is the chart of FAB-MS.

(Analysis of Ingredient F)

Based on the above analysis, Ingredient F was proved to be capsanthin monoester. The kinds of its fatty acid and the component ratio in this procedure were proved as shown in Table 5. Its spectrum data is as follows:

UV-VIS (ether) λ:468, 496 nm, ¹H-NMR (CDCl₃) δ:0.86 (3H, s, H-16′), 0.88 (3H, t, J=7 Hz, CH₃ fatty acid), 1.07 (6H, s, H-16, 17), 1.18 (3H, s, H-17′), 1.25 (s, CH₂ fatty acid), 1.32 (6H, s, H-18′), 1.48 (1H, dd, J=12, 12 Hz, 2β), 1.57 (1H, dd, J=15, 3.5 Hz, H-4′β), 1.74 (1H, dd, J=13.5, 4 Hz, H-2′β), 1.74 (3H, s, H-18), 1.77 (1H, ddd, J=12, 4, 1.5 Hz, H-2 α), 1.96 (3H, s, H-19′), 1.97 (6H, s, H-19, 20), 1.99 (3H, s, H-20′), 2.09 (1H, dd, J=13.5, 8 Hz, H-2′α), 2.04 (1H, dd, J=15.5, 11 Hz, H-4β), 2.39 (1H, ddd, J=15.5, 5.5, 1.5 Hz, H-4α), 2.27 (2H, t, 7 Hz, CH₂ fatty acid), 2.99 (1H, dd, J=15, 9 Hz, H-4′α), 4.00 (1H, m, H-3), 5.24 (1H, m, H-3, 3′), 6.13 (2H, d, AB-type, H-7, 8), 6.16 (1H, d, J=11 Hz, H-10), 6.23 (1H, d, J=10.5, H-14), 6.36 (1H, d, J=15 Hz, H-12), 6.36 (1H, d, J=11 Hz, H-14′), 6.44 (1H, d, J=15 Hz, H-7′), 6.55 (1 H, d, J=15 Hz, H-12′), 6.59 (1H, d, J=11 Hz, H-10′), 6.64 (1H, dd, J=15, 11 Hz, H-11), 6.68 (1H, dd, J=15, 11 Hz, H-11′), 6.70 (2H, m, H-15, 15′), 7.34 (1H, d, J=15 Hz, H-8′), ¹³C-NMR (CDCl₃) δ:12.74 (C-19, 20), 12.80 (C-20′), 12.87 (C-19′), 14.13 (CH₃ fatty acid), 20.78 (C-18′), 21.63 (C-18), 22.69 (CH₂ fatty acid), 24.77 (CH₂ fatty acid), 25.05 (C -17′), 25.61 (C-16′), 28.72 (C-16), 29.14, 29.27, 29.35, 29.47, 29.60, 29.64 (CH₂ fatty acid), 30.26 (C-17), 31.91, 34.65 (CH₂ fatty acid), 37.12 (C-1), 42.20 (C-4′), 42.54 (C-4), 43.73 (C-1′), 47.66 (C-2′), 48.40 (C-2), 58.51 (C-5′), 65.08 (C-3), 73.24 (C-3′), 120.80 (C-7′), 124.05 (C-11), 125.51 (C-7), 125.84 (C-5), 124.60 (C-11′), 131.20 (C-10), 131.21 (C-15), 132.35 (C-13), 133.95 (C-9′), 134.99 (C-14′), 135.87 (C-9), 136.11 (C-14), 136.94 (C-13′), 137.60 (C-12), 137.71 (C-6), 138.41 (C-8), 140.70 (C-10′), 141.82 (C-12′), 147.02 (C-8′), 173.63 (C═O fatty acid), 202.51 (C-6′), FAB-MS m/z:822 (M⁺) for C₅₆H₈₂O₄ (capsanthin-3′-palmitate), 794 (M⁺) for C₅₄H₈₂O₄ (capsanthin-3′-myristate), 766 (M⁺) for C₅₂H₇₈O₄ (capsanthin-3′-laurate), the ester component ratio of capsanthin-3′-ester; palmitate: myristate:laurate (12:70:18)

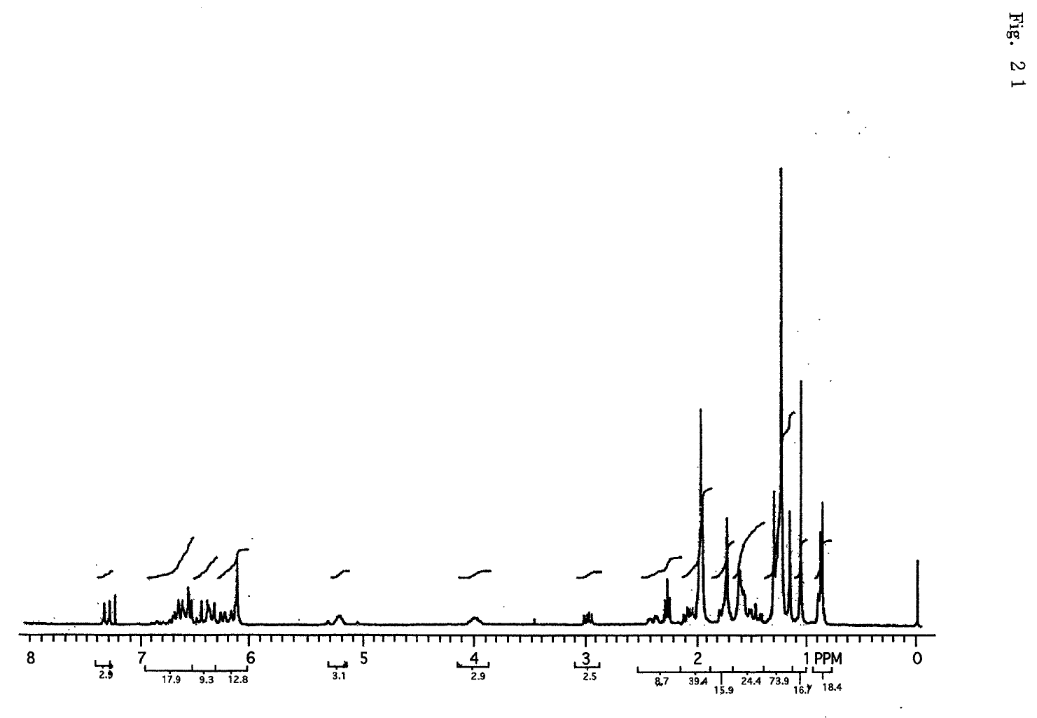

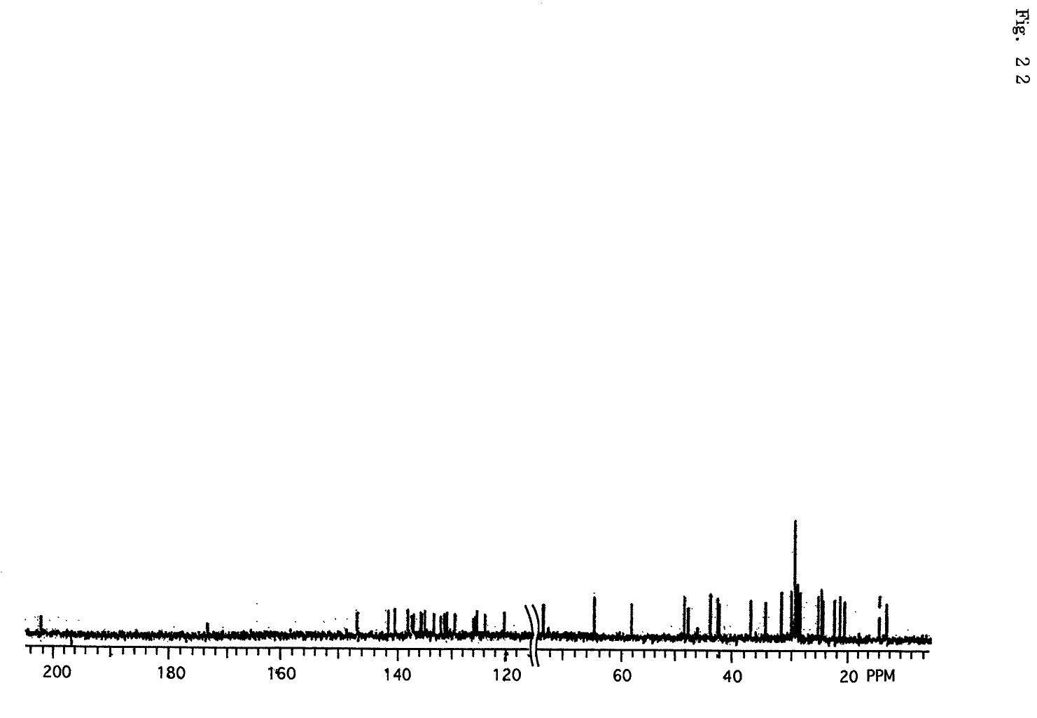

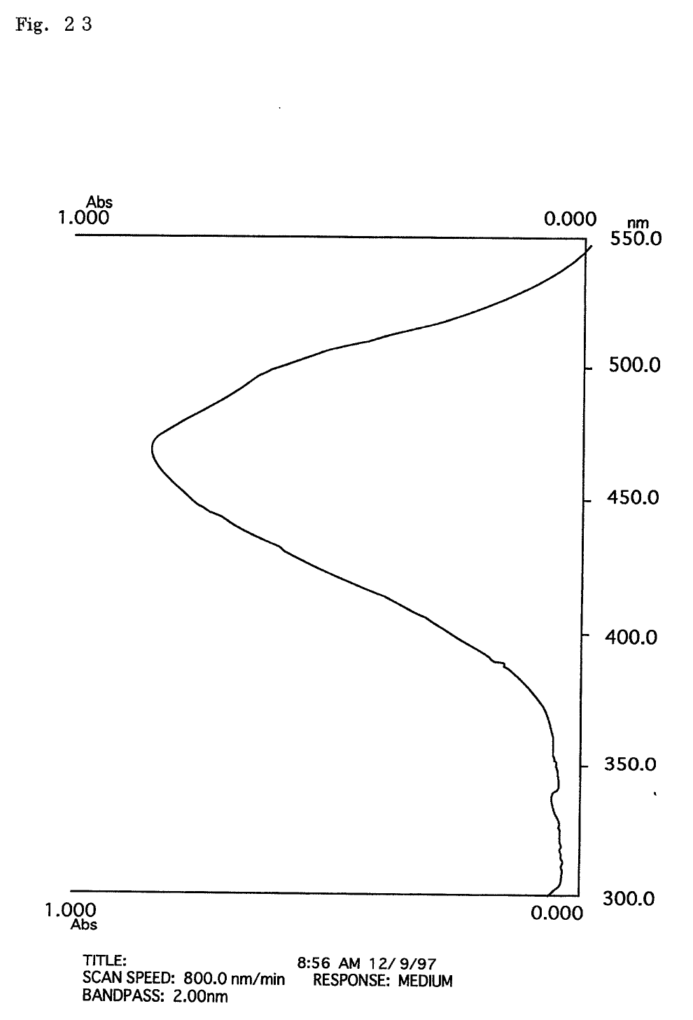

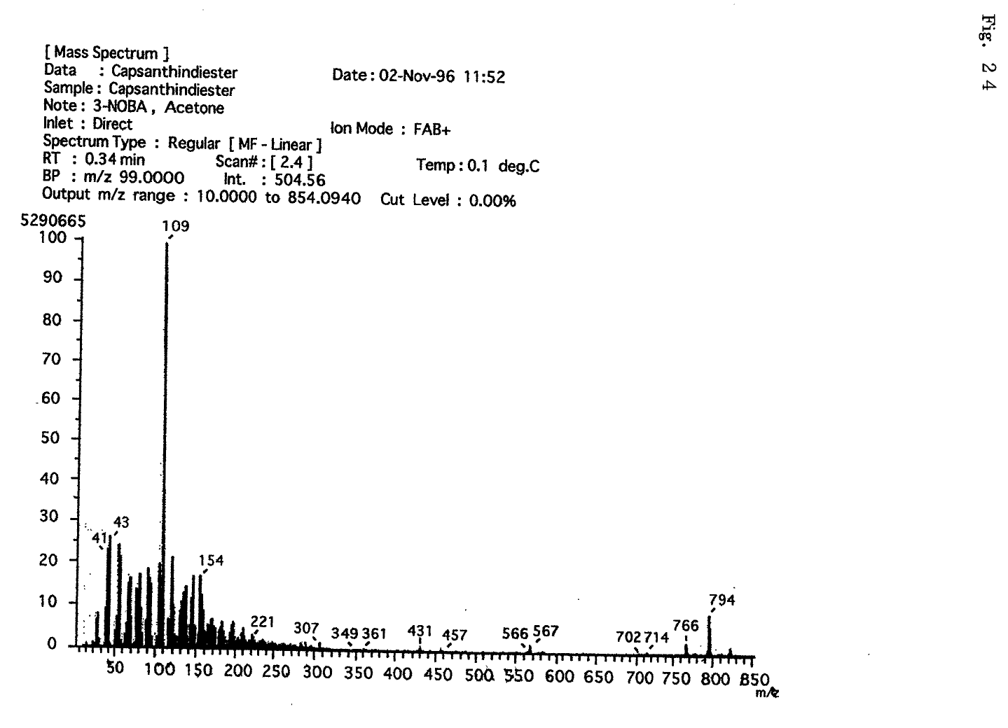

FIG. 21 is the chart of ¹H-NMR of capsanthin monoester, and FIG. 22 is the chart of ¹³C-NMR. Further, FIG. 24 is the chart of FAB-MS.

TABLE 5

| | Molecular | |
|---|---|---|
| Component of Fatty Acid | weight | Component Ratio |
| Palmitic | 822 | 12% |
| Myristic | 794 | 70% |
| Lauric | 766 | 18% |

(Analysis of Ingredient G)

Based on the above-mentioned analysis, Ingredient G was proved to be capsanthin. Its spectrum data is as follows:

UV-VIS (ether) λ:468, 496 nm, ¹H-NMR (CDCl₃) δ:0.84 (3H, s, H-16′) 1.07 (6H, s, H-16, 17), 1.21 (3H, s, H-17′) 1.37 (6H, s, H-18′), 1.48 (1H, dd, J=12, 12 Hz, 2β), 1.49 (1H, dd, J=15, 3.5 Hz, H-4′β), 1.71 (1H, dd, J =13.5, 4 Hz, H-2′β), 1.74 (3H, s, H-18), 1.7 7 (1H, ddd, J=12, 4, 1.5 Hz, H-2α), 1.96 (3H, s, H-19′), 1.97 (6H, s, H-19, 20), 1.99 (3H, s, H-20′), 2.00 (1H, dd, J=13.5, 8 Hz, H-2′α), 2.04 (1H, dd, J=15.5, 11 Hz, H -4β), 2.39 (1H, ddd, J=15.5, 5.5, 1.5 Hz, H-4′), 2.96 (1H, dd, J=15, 9 Hz, H-4′), 4.00 (1H, m, H-3), 4.51 (1H, m, H-3, 3′), 6.13 (2H, d, AB-type, H-7, 8), 6.16 (1H, d, J=11 Hz, H-10), 6.23 (1H, d, J=10.5, H-14), 6.36 (1H, d, J=15 Hz, H-12), 6.36 (1H, d, J=11Hz, H-14′), 6.44 (1H, d, J=15 Hz, H-76.55 (1H, d, J=15 Hz, H-12′),6.59 (1H, d, J=11 Hz, H-10′), 6.64 (1 H, dd, J=15, 11 Hz, H-11), 6.68 (1H, dd, J=15 11 Hz, H-11′), 6.70 (21H, m, H-15, 15′), 7.34 (1H, d, J=15 Hz, H-8′), ¹³C-NMR (CDCl₃) δ: 12.78 (C-19, 20), 12.75 (C-20′), 12.88 (C-19′), 21.39 (C-18′), 21.63 (C-18), 25.20 (C -17′), 25.90 (C-16′), 28.72 (C-16), 30.26 (C-17), 37.12 (C-1), 42.50 (C-4), 43.97 (C -1′), 45.40 (C-4′), 48.69 (C-2), 50.93 (C-2′), 58.93 (C-5′),65.08 (C-3), 70.39 (C-3′), 120.80 (C-7′),124.05 (C-11), 125.51 (C7), 126.20 (C-5), 124.60 (C-11′), 131.20 (C-10), 131.21 (C-15), 132.35 (C-13), 133.95 (C-9′), 134.99 (C-14′), 135.87 (C-9), 136.11 (C-14), 136.94 (C-13), 137.60 (C-12), 137.81 (C-6), 138.41 (C-8), 140.70 (C-10′), 141.82 (C-12′), 147.02 (C-8′), 202.51 (C-6′), FAB-MS m/z:584 (M⁺) for C₄₀H₅₆O₃

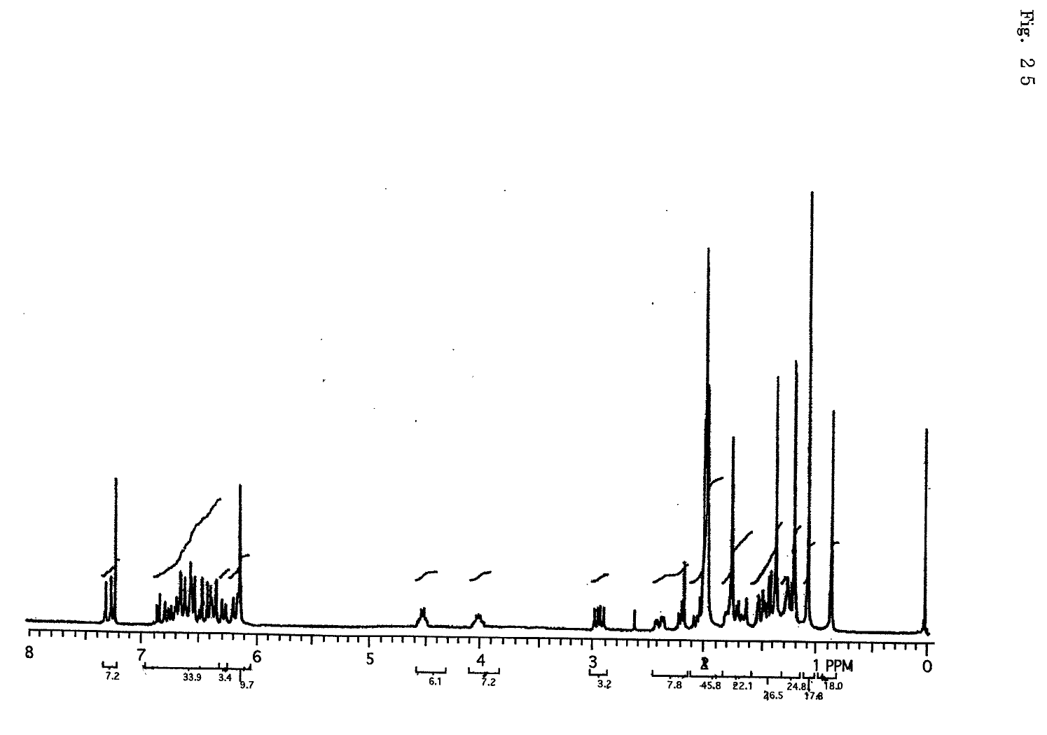

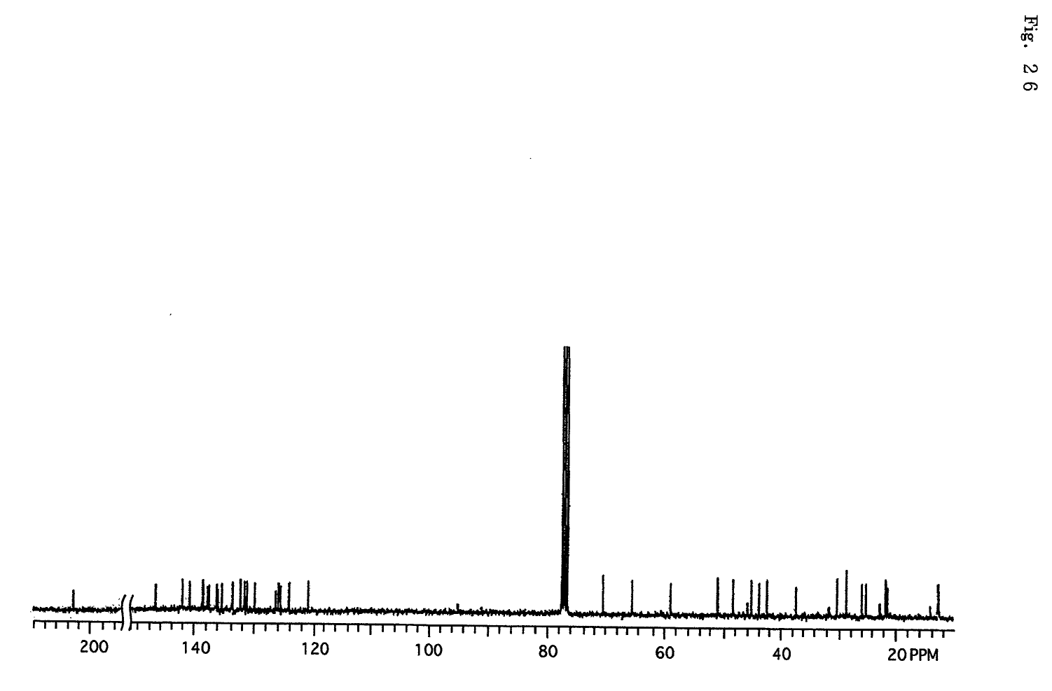

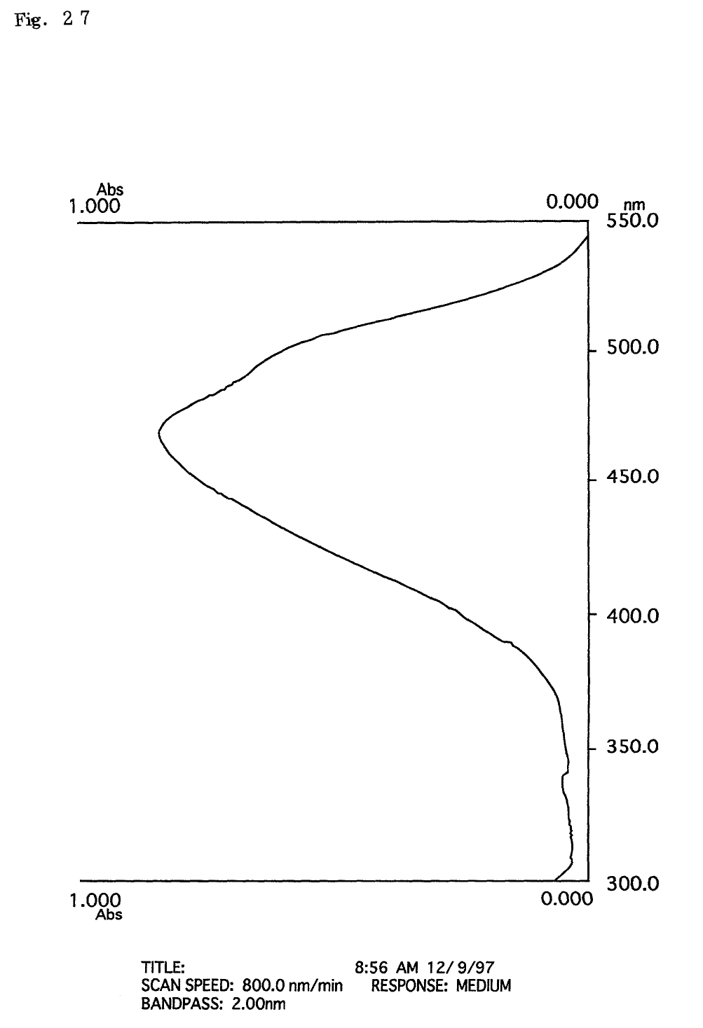

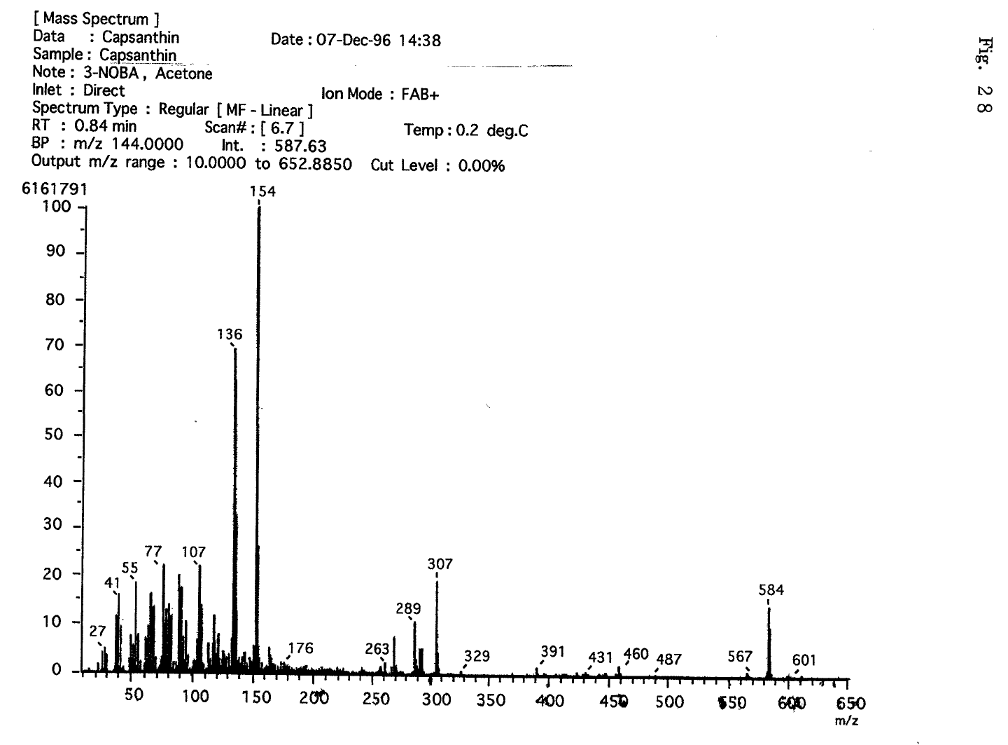

FIG. 25 is the chart of ¹H-NMR of capsanthin, and FIG. 26 is the chart of ¹³C-NMR. Further, FIG. 27 is the chart of UV-VIS and FIG. 28 is the chart of FAB-MS.

Above described capsanthin, capsanthin monoester, capsanthin diester, capsorubin diester, capsanthin 3,6-epoxide, capsorubin and cucurbitaxanthin A-3′ ester are classified as the family of carotenoids and each has a structural diagram as illustrated in the following. In case these carotenoids are esters, their fatty acid R₁ and R₂ are not specified particularly. In case that these carotenoids are extracted from paradicsom paprika, the kinds of component fatty acid are described in the above tables.

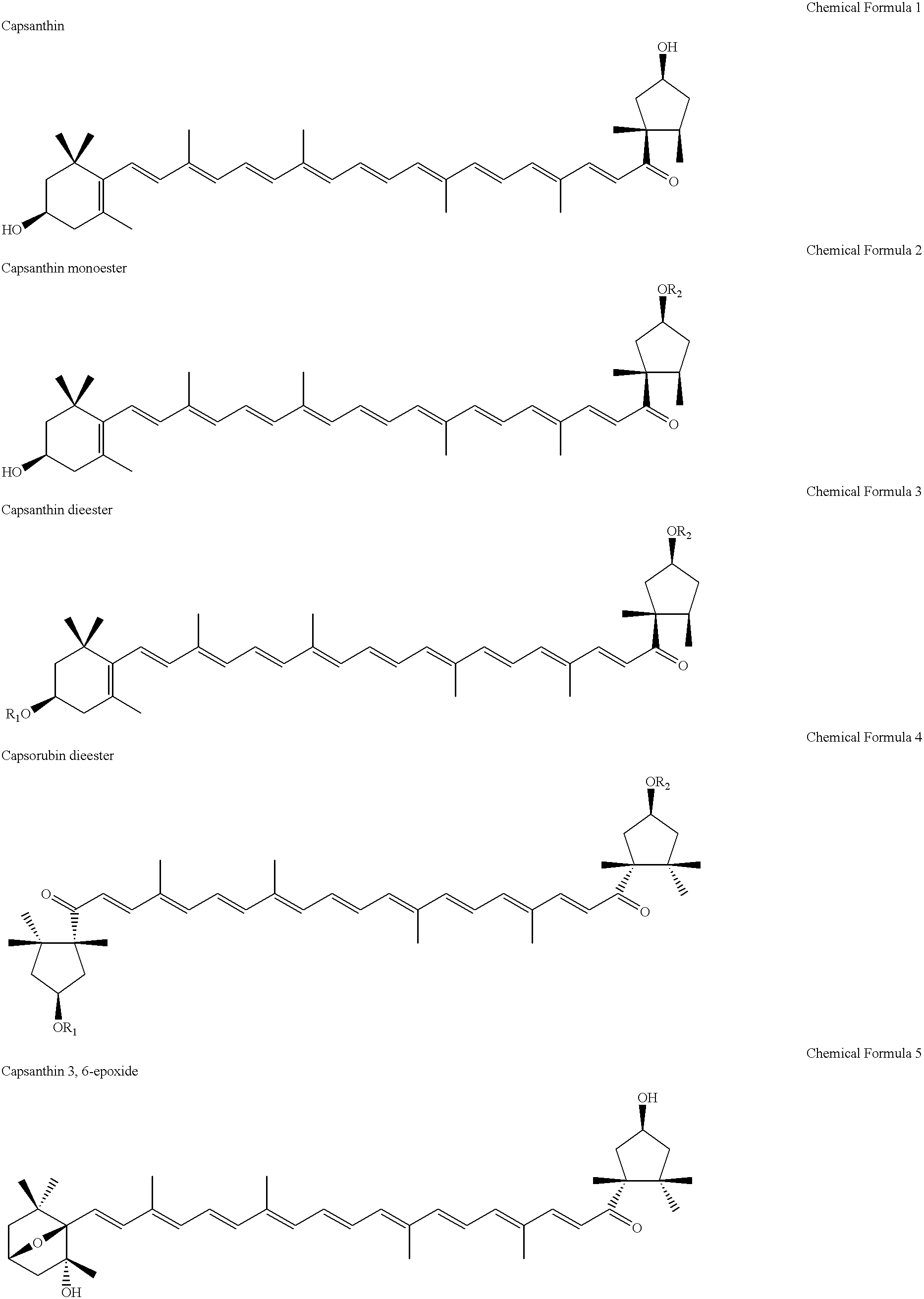

(Measurement of inactivation of Epstein-Barr virus genome incidence)

The inactivation of Epstein-Barr virus genome incidence using these carotenoids was measured under the condition as follows. PRMI1640 added embryonic serums and antibiotics are used as the culture liquid of Raji cells, virus latent infection-human lymphotropic bulbous cells. Under this culture conditions, the rate of natural incidence of Epstein-Barr virus of early antigens are not more than 0.1%. Raji cells, adjusted at the concentration of 1×10⁶ cell/ml is cultured for 48 hours at 37° C. in the above described culture liquid after adding 4 mM of n-butyric acid, 20 ng/ml of TPA and 1000 Mol ration/TPA(20 ng=32 pmol/ml) of test substance. Then cells which generated Epstein-Barr virus early antigens are detected in the indirect immunofluorescence method using serums from epipharynx cancer patients. The ratio of positive cells calculated against the control without adding test_substance is regarded as inactivation of virus genome incidence. In addition, the same inactivation were measured as varying the concentration of test substance to 500 Mol ratio/TPA(20 ng=32 pmol/ml), 100 Mol ratio/TPA(20=32 pmol/ml) and 10 Mol ratio/TPA(20 ng=32 pmol/ml). The results are shown in the Table 6 below.

TABLE 6

| Concentration¹⁾ | 1000 | 500 | 100 | 10 |
|---|---|---|---|---|
| Capsanthin²⁾ | 91.4(70) | 59.7 | 17.6 | 0 |
| Capsanthin monoester²⁾ | 96.8(70) | 68.4 | 26.6 | 4.1 |
| Capsanthin diester²⁾ | 100(70) | 75.5 | 30.7 | 9.6 |
| Capsorubin diester²⁾ | 100(70) | 73.9 | 28.0 | 7.2 |
| Cucurbitaxanthin-A-3′-ester²⁾ | 100(70) | 61.0 | 13.8 | 0 |
| Capsantlim 3, 6-epoxide²⁾ | 100(70) | 67.2 | 20.8 | 5.4 |
| Capsorubin²⁾ | 100(70) | 64.1 | 19.9 | 0 |
| β-Carotene²⁾ | 97.5(70) | 75.0 | 10.6 | 0 |

Unit
¹⁾Mol ratio/TPA(20 ng = 32 pmol/ml)
²⁾% inhibitory rate(% survival rate of Raji cells)

The above described carotenoids extracted from paradicsom paprika showed strong inactivate effect of carcinogenesis virus in almost the same or higher level of β-carotene which is known as an carcinogenesis inhibitory promoter. Survival rates of Raji cells are sustained at least 70% and no particular virulence against cells was observed. Accordingly, the above-mentioned carotenoids were proved to have the carcinogenesis inhibitory effect and can be used as active ingredients of carcinogenesis inhibitor.

(Two-stage carcinogenesis inhibitory test on mouse skin)

As mentioned above, it is confirmed that these carotenoids show strong inactivate effect against carcinogenesis virus. Now, skin cancer inhibitory effects were tested on mice to define carcinogenesis inhibitory effects of capsanthin, capsanthin monoester, and capsanthin diester. Conditions of this Two-stage carcinogenesis inhibitory test on mouse skins were as follows:

Body hair of one group of 15 ICR female mice (age 6 weeks) on their back were shaved and after 24 hours, applied (100 μMg, 390 nmol) of 7,12-dimethylbenz[α]anthracene (hereinafter called “DMBA”) dissolved in acetone (0.1 ml) on their shaved skin of back as an initiation. Since one week later, each group of mice had been treated as follows:

First group: TPA (1 μg, 1.7 nmol) dissolved in acetone (0.1 ml) had been applied for 20 consecutive weeks, twice a week, as a promotion. During this treatment, additional acetone (0.1 ml) was applied on the same part one hour prior to each TPA application. (positive control group).

Second-Fourth group: As the first group, TPA (1 μg, 1.7 nmol) dissolved in acetone (0.1 ml) had been applied for 20 consecutive weeks, twice a week, as a promotion but 85 nmol of test substance (capsanthin group) dissolved in acetone(0.1 ml) (separated by removing methanol extracts solvent), was applied one hour prior to each application instead of additional acetone. Each test substance was for Second group: Capsanthin, Third group: Capsanthin monoester, Fourth group: Capsanthin diester

The Two-stage carcinogenesis inhibitory test on mouse skins were as follows:

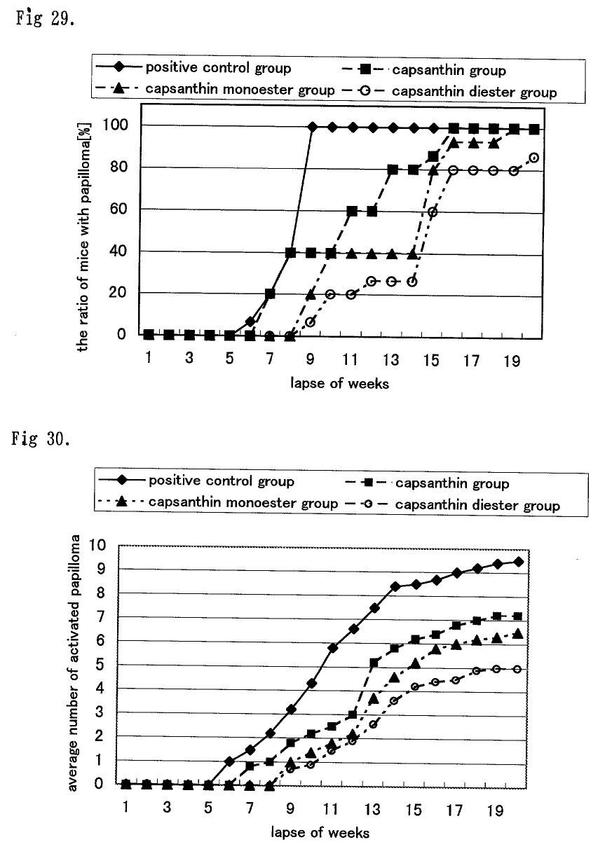

Until the 20^{th} week since the start of a promotion by TPA application, incidence of papilloma on back of mice had been observed once a week. And the rate of mice with papilloma incidence and the average number of papilloma incidence per mouse were evaluated based on the comparison of the positive control group and the second group. The results are shown in FIG. 29 and 30.

As the results shown in FIG. 29, the first tumor was formed on the 7^{th} week after the start of the promotion and by the 11^{th} week, tumors were found on all of the mice in the positive control group. In the second group (capsanthin treatment group), the first tumor was formed on the 7^{th} week and in the third group (capsanthin monoester treatment group) and forth group (capsanthin diester), on 9^{th} week, in the which proved its effects to delay tumor formation. Moreover, 13.3% of mice in the forth group was still free from tumor formation at the end of this 20-week experiment.

Further, as FIG. 30 shows, the average number of tumors per mouse in the positive control group after 20 weeks was 9.1 but was 7.2 in the second group, 6,5 in the third group and 4.5 in the forth group which proved its approximately 23%, 31% and 45% of carcinogenesis inhibitory effects respectively.

From the above-mentioned results, capsanthin, capsanthin monoester and capsanthin diester were proved to contain carcinogenesis inhibitory effects in the Two-stage carcinogenesis inhibitory test on mouse skin.

Industrial Applicability

This invention provides excellent and natural carcinogenesis inhibitors, such as the one containning Epstein-Barr virus inactivating effects. Accordingly, the carcinogenesis inhibitors and extracts from paradicsom paprika of the present invention can be expected to effect as an anti-carcinogenesis and can be applied in various ways in each field of medical, cosmetic and health-food industries.

1. Carcinogenesis inhibitor comprising capsanthin as an active ingredient.

2. Carcinogenesis inhibitor comprising capsanthin esters as an active ingredient.

3. Carcinogenesis inhibitor as set forth in claim 2, comprising capsanthin monoester as an active ingredient.

4. Carcinogenesis inhibitor as set forth in claim 2, comprising capsanthin diester as an active ingredient.

5. Carcinogenesis inhibitor as set forth in claim 2 wherein at least one of fatty acid composing capsorubin esters is selected from palmitic acid, lauric acid or myristic acid.

6. Carcinogenesis inhibitor comprising capsorubin diester as an active ingredient.

7. Carcinogenesis inhibitor as set forth in claim 6 wherein at least one of fatty acid composing capsorubin ester is selected from palmitic acid, lauric acid or myristic acid.

8. Carcinogenesis inhibitor comprising cucurbitaxanthin-A-3′-ester as an active ingredient.

9. Carcinogenesis inhibitor as set forth in claim 8 wherein cucurbitaxanthin-A-3′-ester is selected from palmitic acid ester, lauric acid ester or myristic acid ester.

10. Carcinogenesis inhibitor comprising capsanthin 3,6-epoxide as an active ingredient.

11. (Canceled)

12. Carcinogenesis inhibitor comprising at least two active ingredients selected from capsanthin ester, capsorubin diester, cucurbitaxanthin-A-3′-ester and capsanthin 3,6-epoxide.

13. Carcinogenesis inhibitor comprising capsanthin monoester and capsanthin diester as active ingredients.

14. Carcinogenesis inhibitor comprising extracts from paradicsom paprika comprising at least one active ingredient selected from capsanthin ester, capsorubin diester, cucurbitaxanthin-A-3′-ester and capsanthin 3 6-epoxide.

15. (Canceled)

16. (Canceled)

17. Plant extracts from paradiscom paprika with carcinogenesis inhibitory effects comprising at least one active ingredient selected from capsanthin ester, capsorubin diester, cucurbitaxanthin-A-3′-ester and capsanthin 3,6-epoxide.

18. (Canceled)

19. Plant extracts from paradicsom paprika with carcinogenesis inhibitory effects comprising capsanthin monoester and capsanthin diester as active ingredients.

20. Plant extracts from paradicsom paprika comprising capsanthin as an active ingredient for inhibiting carcinogenesis.

21. A cosmetic product comprising at least one active ingredient for inhibiting carcinogenesis selected from capsanthin ester, capsorubin diester, cucurbitaxanthin-A-3′-ester and capsanthin 3,6-epoxide.

22. A cosmetic product comprising at least one element from plant extracts selected from capsanthin ester, capsorubin diester, cucurbitaxanthin-A-3′-ester and capsanthin 3,6-epoxide as an active ingredient for inhibiting carsinogenesis which are contained in paradiscom paprika.

23. A cosmetic product comprising at least one element extracted from paradiscom paprika selected from capsanthin ester, capsorubin diester, cucurbitaxanthin-A-3′-ester and capsanthin 3,6-epoxide as an active ingredient for inhibiting carsinogenesis.

24. A food product compriosing at least one active ingredient for inhibiting carcinogenesis selected from capsanthin ester, capsorubin diester, cucurbitaxanthin-A-3′-ester and capsanthin 3,6-epoxide.

25. A food product comprising at least one element from plant extracts selected from capsanthin ester, capsorubin diester, cucurbitaxanthin-A-3′-ester and capsanthin 3,6-epoxide as an active ingredient for inhibiting carsinogenesis which are contained in paradiscom paprika.

26. A food product comprising at least one element extracted from paradiscom paprika selected from capsanthin ester, capsorubin diester, cucurbitaxanthin-A-3′-ester and capsanthin 3,6-epoxide as an active ingredient for inhibiting carsinogenesis.

27. Carcinogenesis inhibitor comprising at least two active ingredients selected from capsanthin, capsanthin ester, capsorubin diester, cucurbitaxanthin-A-3′-ester and capsanthin 3,6-epoxide for inhibiting carcinogenesis.

28. Carcinogenesis inhibitor comprising at least two elements from plant extracts selected from capsanthin, capsanthin ester, capsorubin diester, cucurbitaxanthin-A-3′-ester and capsanthin 3,6-epoxide as an active ingredient for inhibiting carsinogenesis which are contained in paradiscom paprika.

29. Carcinogenesis inhibitor comprising at least two elements extracted from paradiscom paprika selected from capsanthin, capsanthin ester, capsorubin diester, cucurbitaxanthin-A-3′-ester and capsanthin 3,6-epoxide as an active ingredient for inhibiting carsinogenesis.

30. Plant extracts comprising at least two elements extracted from paradiscom paprika selected from capsanthin ester, capsorubin diester, cucurbitaxanthin-A-3′-ester and capsanthin 3,6-epoxide as an active ingredient for inhibiting carsinogenesis.

31. A cosmetic product comprising at least two active ingredients selected from capsanthin, capsanthin ester, capsorubin diester, cucurbitaxanthin-A-3′-ester and capsanthin 3,6-epoxide for inhibiting carsinogenesis.

32. A cosmetic product comprising at least two elements from plant extracts selected from capsanthin, capsanthin ester, capsorubin diester, cucurbitaxanthin-A-3′-ester and capsanthin 3,6-epoxide as an active ingredient for inhibiting carsinogenesis which are contained in paradiscom paprika.

33. A cosmetic product comprising at least two elements of plant extracts from paradiscom paprika selected from capsanthin, capsanthin ester, capsorubin diester, cucurbitaxanthin-A-3′-ester and capsanthin 3,6-epoxide as an active ingredient for inhibiting carsinogenesis.

34. A food product comprising at least two elements of plant extracts selected from capsanthin, capsanthin ester, capsorubin diester, cucurbitaxanthin-A-3′-ester and capsanthin 3,6-epoxide as an active ingredient for inhibiting carsinogenesis.

35. A food product comprising at least two elements from plant extracts selected from capsanthin, capsanthin ester, capsorubin diester, cucurbitaxanthin-A-3′-ester and capsanthin 3,6-epoxide as an active ingredient for inhibiting carsinogenesis which are contained in paradiscom paprika.

36. A food product comprising at least two elements of plant extracts from paradiscom paprika selected from capsanthin, capsanthin ester, capsorubin diester, cucurbitaxanthin-A-3′-ester and capsanthin 3,6-epoxide as an active ingredient for inhibiting carsinogenesis.
